(19) United States
(12) Patent Application Publication
(10) Pub. No.: US 2023/0024099 A1
(43) Pub. Date: Jan. 26, 2023

(54) NEW STRAINS OF LACTIC ACID BACTERIA, FOOD COMPOSITION COMPRISING THEM, PREPARATION OF SUCH COMPOSITION

(71) Applicant: CELERY S.R.L., Polignano a Mare (BA) (IT)

(72) Inventors: Carlo Giuseppe Rizzello, Bari (IT); Susanna Raho, Brindisi (IT); Cinzia Dingeo, Ruvo di Puglia (BA) (IT); Vito Emanuele Carofiglio, Polignano a Mare (BA) (IT); Domenico Centrone, Polignano a Mare (BA) (IT)

(73) Assignee: CELERY S.R.L., Polignano a Mare (BA) (IT)

(21) Appl. No.: 17/756,908

(22) PCT Filed: Jan. 21, 2021
(86) PCT No.: PCT/IB2021/050451

§ 371 Date: Jun. 6, 2022

(30) Foreign Application Priority Data
Jan. 22, 2020 (IT) 102020000001231

(51) Int. Cl. C12N 1/20; A23L 33/135; A23L 7/10; A23L 11/00; A23L 5/10

(52) U.S. Cl. CPC C12N 1/205; A23L 33/135; A23L 7/198; A23L 11/05; A23L 5/13

(57) ABSTRACT

The present invention relates to two new strains of lactic acid bacteria, Lactobacillus brevis DSM 33325 and Lactobacillus plantarum DSM 33326, to a new food composition, and to the process for the preparation thereof.

### FIELD OF THE INVENTION

The present invention relates to two new strains of lactic acid bacteria, to a new food composition, and to its preparation process. In particular, the present invention relates to a new food composition produced exclusively with vegetable ingredients (vegan), not containing lactose and gluten, high in fiber and protein, characterized by a low glycemic index, high protein digestibility, containing such new strains in living and viable form and with high cellular density, and to the preparation process of such a food composition.

### BACKGROUND OF THE INVENTION

In the last decade, scientific research has been directed towards the development of foods, called functional, which bring one or more positive effects to the organism beyond their nutritional value, but modulating a specific physiological function.

The concept of functional food is combined with the current sensitivity of consumers on the issue of diet-health correlation. Recent statistics show, in fact, that the wholesomeness of food currently represents one of the main factors capable of influencing the purchase of a food. Although the concept of “functional” was introduced in Japan as early as the early 1980s, Europe had to wait until the 1990s to develop a real definition recognized by the scientific world. Specifically, following the FUFOSE (Functional Food Science in Europe) project coordinated by the International Life Science Institute (ILSI Europe), the meaning of functional food was unambiguously defined (Serafini et al., 2012, Functional foods: traditional use and European legislation, Int J Food Sci Nutr 63 S1:7-9). According to what was established within the FUFOSE project, “a food can be considered functional if it satisfactorily demonstrates that it has positive and targeted effects on one or more specific functions of the organism, which go beyond the normal nutritional effects, so such that it is relevant for improving health and well-being and/or for reducing the risk of disease. It being understood that functional foods must continue to be foods and must demonstrate their action in the quantities in which they are normally taken in the diet” (Serafini et al. 2012). In this document it is also made clear that the beneficial contributions to the basic food may be due to chemical, enzymatic or biotechnological actions. The benefits of health and medical properties related to functional foods have raised the interest of researchers, consumers and the food industry. Functional foods can come in various types of formulations which include solid, semi-liquid and liquid foods. The category that appears to be most promising is represented by beverages. The latter, in fact, are able to better satisfy the demands of consumers, as they can be easily distributed, stored in refrigerated conditions and allow nutrients and bioactive compounds to be easily incorporated. Although yoghurts and fermented milks are the functional beverages mainly requested by consumers, there is a growing demand for beverages with a formulation similar to yoghurt, but not based on milk, defined as “yoghurt-like beverages”. Among the reasons that push the demand for this new type of food are vegetarianism, the increase in the prevalence of cases of lactose intolerance and hypercholesterolemia. Not least, another negative effect related to excessive consumption of dairy products is exposure to pesticides and hormones (Davoodi et al. 2013, Effects of milk and milk products consumption on cancer: a review. Comprehensive reviews in Food science and Food safety 12:249-264). All of this has led to the development of functional non-alcoholic, non-milk-based beverages that can also act as a vehicle for probiotic microorganisms. Worldwide, several beverages with these characteristics have developed, mainly based on cereals (Kandylis et al., 2016. Dairy and non-dairy probiotic beverages. Current Opinion in Food Science, 7:58-63). In fact, cereals represent the most important resource in terms of carbohydrates, proteins, vitamins, minerals and fibers. Thanks to their health properties, cereals are often used alone or mixed with other ingredients in order to develop new foods. For example, a cereal often used as a basic ingredient in yoghurt-like beverages is rice, as it can determine excellent rheological properties following a gelatinization process (Blandino et al., 2003. Cereal-based fermented foods and beverages. Food Research International, 36:527-543).

Although the technologies proposed experimentally for the production of yoghurt-like cereal-based beverages are different, fermentation is the process most used to improve the food matrix in a natural way from a nutritional, sensorial and shelf life point of view (Hugenholtz, 2013, Traditional biotechnology for new foods and beverages, Curr Opin Biotechnol 24:155-159). In general, in industrial production, the fermentation process is made controllable and reproducible through the use of specific starter cultures, able, once inoculated into the matrix, to grow and modify the characteristics of the food with their metabolism. Lactic acid bacteria (LAB) are used for this purpose in various food substrates such as milk, meat, vegetables and cereals, also because most of them have been recognized as completely safe from a hygienic-sanitary point of view.

The beneficial contributions to the fermentation of which the LAB are responsible in the cereal matrices are different: (a) thanks to the high proteolytic activity during fermentation (Coda et al., 2014, Sourdough lactic acid bacteria: exploration of non-wheat cereal-based fermentation, Food Microbiol 37:51-58), they favor the digestibility of proteins producing peptides and/or amino acids starting from them; (b) they increase the bioavailability of minerals by degrading phytic acid by activating endogenous and/or microbial phytases (Coda et al., 2014); (c) they make other nutrients bioaccessible (e.g. polyphenols and fibers) (Coda et al. 2015, Bran bioprocessing for enhanced functional properties, Current Opinion in Food Science, 1:50-55); (d) through the process of biological acidification they are able to lower the glycemic index (De Angelis et al., 2009, Sourdough fermentation as a tool for the manufacture of low-glycemic index white wheat bread enriched in dietary fiber. European Food Research and Technology, 4:593-601); (e) they positively affect the shelf life of food through the achievement of acidic pH values and through the release of antimicrobial compounds (Gupta et al., 2010. Process optimization for the development of a functional beverage based on lactic acid fermentation of oats, Biochemical Engineering Journal, 52:2:199-204); and (f), finally, they improve the organoleptic qualities. Furthermore, among the advantages reported in the literature there are also an increase in the bioavailability of B vitamins, some amino acids and other compounds with functional activity such as γ-amino butyric acid (GABA) and biogenic peptides (Coda et al., 2010. Use of sourdough fermentation and pseudo-cereals and leguminous flours for the making of a functional bread enriched of gamma-aminobutyric acid (GABA), Int J Food Microbiol, 137:236-245). The fermented matrix can also be a carrier of viable probiotic microorganisms with high cell density (Nagpal et al., 2012, Probiotics, their health benefits and applications for developing healthier foods: a review, FEMS Microbiol Lett 334:1-15). Another advantage that can be obtained with the use of lactic acid bacteria is the possibility of modifying the visco-elastic characteristics of the matrix (viscosity, softness, coherence), thanks to the production of exopolysaccharides (EPS), which they synthesize through the polymerization of simple sugar subunits such as glucose and fructose. On the basis of what has been described and of the scientific literature, the fermentation process of vegetable matrices represents one of the most suitable tools for improving the flavor and nutritional and functional profile of the same.

### SUMMARY OF THE INVENTION

Based on recent experiences conducted in the field of scientific research on cereal and pseudocereal-based matrices (Coda et al., 2011. Manufacture and characterization of functional emmer beverages fermented by selected lactic acid bacteria, Food Microbiology, 28:526-536; Lorusso et al., 2018, Use of selected lactic acid bacteria and quinoa flour for manufacturing novel yogurt-like beverages; Foods, 7, 51), within the scope of the present invention, the inclusion of a legume-based component in the yoghurt-like product was evaluated. The use of the latter, not yet explored in the yoghurt-like beverage/snack sector, is potentially interesting for a number of reasons.

Firstly, legumes, unlike most cereals, being gluten-free, can be safely consumed by celiacs, whose global prevalence is currently estimated at around 1-2% of the entire population.

Secondly, given that the world consumption of legumes is lower than the recommended dose (McCrory et al., 2010, Pulse Consumption, Satiety, and Weight Management, Advances in Nutrition 1:17-30), food sensitization pushes to reintegrate in the diet this food, as proposed by the Mediterranean diet. According to the provisions of the OMG, legumes are essential components in daily nutrition, as they allow the intake of proteins with a high biological value (composition in essential amino acids complementary to that of cereals), dietary fibers, oligosaccharides, vitamins, minerals and polyphenols. Habitual consumption of these foods is associated with a reduction in the risk of cardiovascular diseases, type 2 diabetes, some types of cancers and obesity conditions. Even legumes, like cereals, lend themselves favorably to a fermentation process, which results in an improvement from a sensorial point of view, but also in a reduction of the so-called anti-nutritional factors (Curiel et al., 2015, Exploitation of the nutritional and functional characteristics of traditional Italian legumes: the potential of sourdough fermentation. International Journal of Food Microbiology, 196:51-61), in which these ingredients are rich. The fermentation of legumes is a tradition in many countries, especially outside Europe. Just think of two fermented legume-based foods produced in Japan: soy sauce and miso.

The present inventors have faced the problem of producing a functional food having solid nutritional bases, consisting of vegetable ingredients, not including ingredients containing lactose and/or gluten, high in fiber and protein, characterized by a low glycemic index and high protein digestibility.

At the same time, the present inventors posed the problem of how to increase the world consumption of legumes.

For this purpose, the present inventors have meticulously selected multiple raw materials on the basis of their nutritional complementarity, including legumes, cereals and specific strains of lactic acid bacteria in order to obtain a food with a high nutritional and functional profile.

The bacterial strains Lactobacillus brevis DSM 33325 and Lactobacillus plantarum DSM 33326 were chosen on the basis of their unique characteristics. As shown in example 1 of the experimental part of the present patent application, such bacterial strains fermented on cereal matrices, for example rice, legumes, for example chickpea or lentils, or pseudo-cereals, for example quinoa, have a greater capacity of growth, acidification, production of free amino acids, degradation of anti-nutritional factors, release of polyphenols and consequent anti-oxidant capacity compared to other strains of lactic acid bacteria tested (see Tables 1-3).

Therefore, a first object of the present invention is a bacterial strain selected from:

Lactobacillus brevis, filed on 14 Nov. 2019 with Deutsche Sammlung von Mikroorganismen und Zellkulturen GmbH and identified with the filing number DSM 33325, and

Lactobacillus plantarum, filed on 14 Nov. 2019 with Deutsche Sammlung von Mikroorganismen und Zellkulturen GmbH and identified with the filing number DSM 33326.

Such bacterial strains are suitable for use as a starter in the preparation of a food composition, preferably in the preparation of a yoghurt-like food composition, or of a dehydrated food composition.

Therefore, a second object of the present invention is the use of Lactobacillus brevis DSM 33325 and/or Lactobacillus plantarum DSM 33326 in the preparation of a food composition.

As will be discussed in detail in examples 2-7 of the following experimental part, the food composition according to the present invention possesses both excellent functional characteristics and solid nutritional bases.

Advantageously, such a composition is produced exclusively starting from vegetable ingredients and is classifiable as “vegan”, in particular it does not contain milk or other ingredients containing lactose. Preferably, such a composition is produced exclusively with gluten-free ingredients.

As described in example 2, such a composition is produced with and contains live and viable lactic acid bacteria of the Lactobacillus brevis and Lactobacillus plantarum species.

In particular, as shown in example 3, with particular reference to Tables 6-11, the food composition according to the present invention may be defined as “high in fiber” and “high in protein” and is preferably characterized by:
a high concentration of free total amino acids (TFAA), and in particular of the functional amino acid GABA (γ-aminobutyric acid);
a high concentration of proteins and a low glycemic index (pGI);
presence of high concentrations of polyphenols and therefore shows high antioxidant activity;
a reduced presence of anti-nutritional compounds.

Furthermore, as shown in example 4, with particular reference to Tables 12 and 13, such a composition has excellent sensory characteristics and excellent microbiological shelf life.

Therefore, a third object of the present invention is a food composition based on at least one gluten-free cereal flour (A) and/or at least one legume flour (B) and water (C), said components (A) and/or (B) being fermented with a starter (D) comprising Lactobacillus brevis DSM 33325 and/or Lactobacillus plantarum DSM 33326.

A fourth object of the present invention is a process for the production of the food composition as defined in the third object of the invention, comprising the following steps:
a) mixing in water (C) the at least one gluten-free cereal flour (A) and the at least one legume flour (B),
b) subjecting the resulting mixture to heat treatment (gelatinization) at a temperature between 65° and 100° C.,
c) cooling the gelatinized mixture to a temperature between 2° and 8° C.,
d) inoculating into the gelatinized mixture, after heating at a temperature between 20° and 40° C., a starter (D) comprising at least one lactic acid bacterium selected from the following species Lactobacillus brevis, Lactobacillus plantarum, Pediococcus acidilactici, Leuconostoc mesenteroides, Lactobacillus rossiae, and mixtures thereof,
e) fermenting the mixture resulting from step d) at a temperature between 20° and 40° C.,
f) cooling the mixture resulting from step e) to a temperature between 2° and 8° C.

A fifth object of the present invention is the food composition obtained by the process as defined in the fourth object of the invention.

A sixth object of the present invention is the use as a food supplement of a dehydrated food composition, preferably freeze-dried, according to the third or fifth object of the invention.

### BRIEF DESCRIPTION OF THE FIGURES

FIG. 1. Flow chart representative of the process for obtaining a yoghurt-like food composition.

FIG. 2. Growth kinetics of the mixed starter L. brevis DSM 33325 and L. plantarum DSM 33326 (Gompertz modeling) analyzed in the chickpea/rice/lentil matrix during 18 hours of incubation at 30° C. as described in example 2.

FIG. 3. Acidification kinetics of the mixed starter L. brevis DSM 33325 and L. plantarum DSM 33326 (Gompertz modeling) analyzed in the chickpea/rice/lentil matrix during 18 hours of incubation at 30° C. as described in example 2.

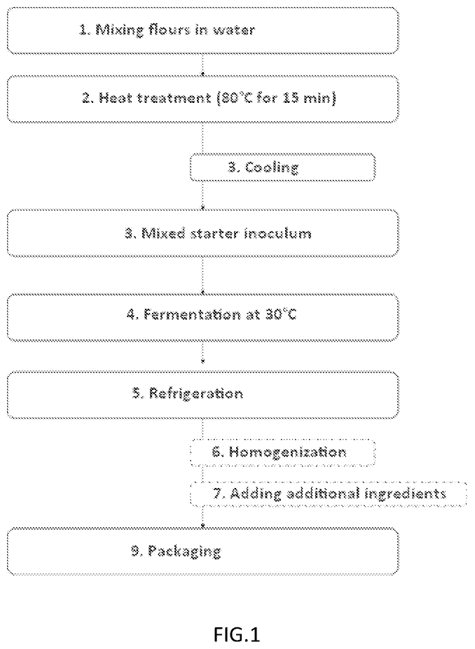

### DETAILED DESCRIPTION OF THE INVENTION

For the purposes of the present description and the appended claims, the following terms and/or expressions in quotation marks are to be understood as defined below.

“Food composition” means any food product intended to be ingested by humans or animals as a source of nutrition and/or calories.

“Yoghurt-like food composition” means a food product similar in viscosity and consistency to yoghurt, but produced with ingredients other than milk, and in this case entirely vegetable. The production process, similar to that of yoghurt, involves fermentation with acidifying lactic acid bacteria which, at the end of the production process, remain alive and viable in the final product at high cell densities.

“Starter” means a culture comprising one or more types of microorganisms used in a live and viable state for the inoculation of food matrices, for transformation by fermentation into ingredients or foods or beverages for food use.

“Alive and viable” referring to microbial cells means microbial cells capable of conducting active metabolism and multiplying.

A first object of the present invention is a bacterial strain selected from:

Lactobacillus brevis, filed on 14 Nov. 2019 with Deutsche Sammlung von Mikroorganismen und Zellkulturen GmbH and identified with the filing number DSM 33325, and

Lactobacillus plantarum, filed on 14 Nov. 2019 with Deutsche Sammlung von Mikroorganismen und Zellkulturen GmbH and identified with the filing number DSM 33326.

A second object of the present invention is the use of Lactobacillus brevis DSM 33325 and/or Lactobacillus plantarum DSM 33326 in the preparation of a food composition, preferably in the preparation of a yoghurt-like food composition (BYL) or of a dehydrated food composition (BYL-dehydrated).

Therefore, a third object of the present invention is a food composition based on at least one gluten-free cereal flour (A) and/or at least one legume flour (B), water (C), and a starter (D) comprising Lactobacillus brevis DSM 33325 and/or Lactobacillus plantarum DSM 33326, preferably in a cell density ratio between 1:1 and 1:10, more preferably 1:1, said components (A) and/or (B) being fermented with said starter (D).

The food composition according to the invention may further comprise one or more additional gluten-free ingredients, for example fruit-based (E), and/or one or more vegetable ingredients (F), and/or one or more vegetable ingredients with a high fiber content (>50% w/w) (G) or high protein content (>50% w/w) (G′), and/or one or more viable probiotic microorganisms (H), and/or at least one salt and/or flavor enhancer (I), and/or at least one sugar and/or sweetener (J), and/or at least one structuring agent (K).

In one embodiment, the food composition is based on at least one gluten-free cereal flour (A) and at least one legume flour (B), water (C) and a starter (D) comprising Lactobacillus brevis DSM 33325 and/or Lactobacillus plantarum DSM 33326, preferably in a cell density ratio between 1:1 and 1:10, more preferably 1:1, said components (A) and (B) being fermented with said starter (D).

In another embodiment, the food composition consists of at least one gluten-free cereal flour (A), at least one legume flour (B), water (C) and a starter (D) comprising Lactobacillus brevis DSM 33325 and/or Lactobacillus plantarum DSM 33326, preferably in a cell density ratio between 1:1 and 1:10, more preferably 1:1, said components (A) and (B) being fermented with said starter (D).

The food composition according to the invention comprises a high concentration in total free amino acids (TFAA), in particular greater than 800 mg/L, more particularly greater than 900 mg/L, and advantageously greater than 1,000 mg/L.

More particularly, the food composition according to the invention comprises a high concentration of the functional amino acid GABA (γ-aminobutyric acid), preferably greater than 100 mg/L.

The food composition according to the invention further comprises a high concentration of proteins, preferably greater than 20% of the energy value of the food composition and a low glycemic index, in particular lower than 55.

In particular, the food composition according to the invention shows an in vitro digestibility value of proteins (IVDP, In Vitro Digestibility Protein) higher than 70%, preferably higher than 75%.

Advantageously, a food composition according to the invention shows a hydrolysis index lower than 30%, preferably equal to or lower than 25%.

The food composition according to the invention comprises high concentrations of polyphenols, in particular greater than 0.20 mmol/L of gallic acid equivalent, preferably greater than 0.25 mmol/L of gallic acid equivalent, and therefore shows high antioxidant activity.

Advantageously, the food composition according to the invention comprises a reduced presence of anti-nutritional compounds, in particular less than 1 mg, preferably less than 0.5 mg of tannins, and/or less than 15 mg, preferably less than 12 mg of raffinose, and/or less than 50 mg, preferably less than 40 mg of phytic acid, and/or less than 40 mg of saponins, preferably less than 35 mg, calculated on 100 mL of food composition.

Advantageously, the food composition according to the invention shows a viscosity higher than 4.00 Pa·s, preferably higher than 4.10 Pa·s.

A fourth object of the present invention is a process for the production of the food composition comprising the following steps:
a) mixing in water (C) the at least one gluten-free cereal flour (A) and the at least one legume flour (B),
b) subjecting the resulting mixture to heat treatment (gelatinization) at a temperature between 65° and 100° C.,
c) cooling the gelatinized mixture to a temperature between 2° and 8° C.,
d) inoculating into the gelatinized mixture, after heating at a temperature between 20° and 40° C., a starter (D) comprising at least one lactic acid bacterium selected from the following species Lactobacillus brevis, Lactobacillus plantarum, Pediococcus acidilactici, Leuconostoc mesenteroides, Lactobacillus rossiae, and mixtures thereof,
e) fermenting the mixture resulting from step d) at a temperature between 20° and 40° C.,
f) cooling the mixture resulting from step e) to a temperature between 2° and 8° C.

The process for the production according to the invention may also include the following optional steps:
g) high-pressure homogenization, and/or
h) addition of further ingredients, suitably pasteurized if necessary, such as one or more fruit-based ingredients (E), and/or one or more vegetable ingredients (F), and/or one or more gluten-free vegetable ingredients with a high fiber content (>50% w/w) (G) or high protein content (>50% w/w) (G′), and/or one or more viable probiotic microorganisms (H), and/or at least one salt and/or flavor enhancer (I), and/or at least one sugar and/or sweetener (J), and/or at least one structuring agent (K), and/or
i) packaging, and/or
j) dehydration by freeze-drying or spray-drying.

In this regard, it is specified that the addition of one or more viable probiotic microorganisms (H) is additive and not substitute with respect to the use of the starter (D).

Preferably, according to the process of the invention, the mixing is carried out mechanically with stirrers, plunger or planetary arm mixers.

Preferably, according to the process of the invention, in the mixture resulting from step a) the percentage by weight of gluten-free cereal flour (A) and/or legume flour (B) is between 5% and 50% with respect to the total volume of the mixture, more preferably between 10% and 35%; advantageously, such a percentage is 20% with respect to the total volume of the mixture.

Preferably, according to the process of the invention, in step b) the heat treatment with the aim of obtaining the gelatinization of the starch fraction and pasteurization, is carried out at a temperature between 75° and 90° C. under stirring, preferably at 40-100 rpm, and preferably has a duration of between 10 and 30 minutes, more preferably of about 15 minutes.

Advantageously, according to the process of the invention, in step b) the mixture reaches 80° C. at its “core”, that is, in the point of the container farthest from the exchange surface thereof (innermost).

Preferably, according to the process of the invention, in step c) the mixture is subjected to cooling at a temperature between 3° and 6° C., advantageously at about 4° C., in a time ranging from 30 seconds to 10 minutes, preferably from 1 to 5 minutes, advantageously about 2 minutes.

Preferably, according to the process of the invention, in step d) the starter (D) is inoculated after bringing the mixture to a temperature between 25° and 35° C., advantageously to about 30° C.

Preferably according to the process of the invention, the starter (D) comprises Lactobacillus brevis and Lactobacillus plantarum; even more preferably, it comprises Lactobacillus brevis DSM 33325 and Lactobacillus plantarum DSM 33326; even more preferably, it comprises Lactobacillus brevis DSM 33325 and Lactobacillus plantarum DSM 33326 in a cell density ratio ranging from 1:1 to 1:10, preferably from 1:1 to 1:5, advantageously of about 1:1.

Preferably, according to the process of the invention, in step e) the fermentation takes place at a temperature between 20° and 35° C., advantageously at about 30° C., preferably for a time between 8 and 24 hours, more preferably between 10 and 20 hours, advantageously 12 hours.

Preferably, according to the process of the invention, in step f) the cooling takes place at a temperature between 3° and 6° C., advantageously at 4° C., more preferably in a time between 5 and 20 minutes, advantageously of about 5 min.

A fifth object of the present invention is the food composition obtained by the process as defined in the fourth object of the invention.

Preferably according to the composition and process of the invention, the gluten-free cereal flour (A) is selected from naturally gluten-free cereal flours, such as for example rice, maize, millet, teff, sorghum, and mixtures thereof; more preferably the gluten-free flour is rice.

Preferably according to the composition and process of the invention, the gluten-free cereal flour (A) comprises: proteins from 5 to 15 g, preferably 8 g; carbohydrates from 30 to 90 g, preferably 70 g; fibers from 2 to 15 g, preferably 3; fats from 0.3 to 8 g, preferably 1.5 g per 100 g of flour (A).

Preferably according to the composition and process of the invention, the legume flour (B) is selected from flours obtained from bean, Phaseolus vulgaris L.; pea, Pisum sativum L.; broad bean, Vicia faba L.; lupine, Lupinus albus; chickpea, Cicer arietinum L.; pigeon pea, Cajanus indicus; peanuts, Arachis hypogaea L.; soy, Glycine max; lentil, Lens culinaris; cicerchia, Lathyrus sativus; carob, Ceratonia siliqua; pseudocereals (amaranth, Amaranthus spp., quinoa, Chenopodium quinoa; buckwheat, Fagopyrum esculentum) and mixtures thereof; more preferably it is selected from flours obtained from Cicer arietinum L., Lens culinaris, Ceratonia siliqua, Chenopodium quinoa and mixtures thereof; even more preferably it is selected from flours obtained from Cicer arietinum L., Lens culinaris, Chenopodium quinoa and mixtures thereof.

Preferably according to the composition and process of the invention, the legume flour (B) comprises: proteins from 5 to 30 g, preferably 20 g; carbohydrates from 5 to 70 g, preferably 60 g; fibers from 2 to 20 g, preferably 10 g; fats from 1 to 50 g, preferably 5 g per 100 g of flour (B).

Preferably according to the composition or the process of the invention, the starter (D) has cell density between 1×10⁷ and 5×10⁷ cfu/ml.

Preferably according to the composition or process of the invention, the starter (D) comprising Lactobacillus brevis DSM 33325 and Lactobacillus plantarum DSM 33326 in a cell density ratio between 1:1 and 1:10, preferably between 1:1 and 1:5, advantageously of about 1:1.

Preferably according to the composition or process of the invention, the starter (D) is in liquid, pellet or freeze-dried form.

Preferably according to the composition or process of the invention, among the additional gluten-free ingredients, the one or more fruit-based ingredients (E) may be selected from: apricot, watermelon, pineapple, orange, avocado, banana, bergamot, carambola, cherry, cherimoya, durian, fig, prickly pear, finger lime, strawberry, jujube, granadilla, Graviola, guava, kiwi, kumquat, raspberry, lime, lemon, litchi, lacuma, almond, mango, mandarin, passion fruit, apple, pomegranate, melon, blueberry, blackberry, coconut, papaya, passion fruit, pear, peach, pitaya, plane tree, pomelo, grapefruit, plum, rambutan, salak, sapodilla, plum, tamarillo, ugli, grape, sapote, preferably apricot, cherry, strawberry, raspberry, mango, pomegranate, blueberry, blackberry, pistachio, and mixtures thereof; such a fruit-based ingredient (E) may be in the formulation of puree, juice, pulp, concentrate, nectar, dehydrated fruit, freeze-dried fruit, fresh pasteurized fruit; preferably puree and dehydrated fruit; such a fruit-based ingredient (E) includes: proteins from 0.1 to 2 g; carbohydrates from 5 to 20 g; fibers from 1.5 to 3 g; fats 0.1 to 1 g per 100 g of fruit-based ingredient.

Preferably according to the composition or the process of the invention, the one or more vegetable ingredient (F) may be selected from ginger, cassava, chestnuts, pistachio, vanilla, ginger, star anise, cinnamon, cocoa, coffee, chocolate grains, hazelnuts, walnuts, macadamia nuts, licorice, cocoa, mint, and mixtures thereof; such a vegetable ingredient (F) may be in the formulation of puree, dehydrated or freeze-dried.

Preferably according to the composition or process of the invention, the one or more vegetable ingredient with a high fiber content (G) may be selected from oat and citrus fibers; the one or more vegetable ingredient with a high protein content (G′) may be selected from soy protein isolates or other legumes, pseudocereals, etc.

Preferably according to the composition or process of the invention, the one or more viable probiotic microorganisms (H) may be selected from: Lactobacillus johnsonii, Lactobacillus casei, Bifidobacterium lactis, Lactobacillus rhamnosus and mixtures thereof.

Preferably according to the composition or process of the invention, the at least one salt and/or flavor enhancer (I) may be selected from NaCl (sodium chloride), glutamic acid and salts thereof, glycine and salts thereof, aspartic acid and salts thereof, guanylic acid and salts thereof, inosinate.

Preferably according to the composition or process of the invention, the at least one sugar and/or sweetener (J) may be selected from glucose/dextrose, fructose, sucrose, maltose, cyclamate, sorbitol, maltitol, mannitol, isomalt, xylitol, saccharin, polyols, aspartame, acesulfame.

Preferably according to the composition or process of the invention, the at least one structuring agent (K) may be selected from exopolysaccharides (EPS), hydrocolloids, gums, carrageenan, agar, modified starches, etc.

In one embodiment, the composition of the invention comprising at least one of the additional gluten-free ingredients (E) to (K).

Preferably according to the composition or the process of the invention, the composition has a pH of between 3.8 and 4.5, preferably pH 4.2 and a cell density greater than 10⁸ cfu/ml; preferably between 1×10⁹ and 7×10⁹ cfu/ml.

In one embodiment, the food composition according to the invention is in yoghurt-like form (BYL), preferably having a viscosity of between 0.001 and 1,000 Pa·s.

In one embodiment, the food composition according to the invention has a viscosity of between 2 and 15 Pa·s, preferably between 4 and 10 Pa·s, for example 4.23 Pa·s (yoghurt-like base food composition (BYL-base) or “base formulation”, with a texture similar to that of a conventional yoghurt).

In another embodiment, the food composition according to the invention has a viscosity of between 5 and 50 mPa·s, preferably between 5 and 20 mPa·s, for example 9.21 mPa·s (yoghurt-like food composition to drink (BYL-to drink) with a texture similar to that of a conventional yoghurt to drink).

In another embodiment, the food composition according to the invention has a viscosity of between 100-800 Pa·s, preferably between 300 and 600 Pa·s, for example 400 Pa·s (yoghurt-like food composition for filling (BYL-for filling).

In a preferred embodiment, the food composition according to the invention is yoghurt-like base (BYL-base).

In another preferred embodiment, the food composition according to the invention is yoghurt-like to drink (BYL-to drink).

In another preferred embodiment, the food composition according to the invention is yoghurt-like for filling (BYL-for filling).

In another preferred embodiment, the food composition according to the invention is dehydrated (BYL-dehydrated), preferably freeze-dried (BYL-lyophile).

A sixth object of the present invention is the use as a food supplement of a dehydrated food composition, preferably freeze-dried, according to the third or fifth object of the invention.

### EXPERIMENTAL PART

### Example 1: Selection of Lactic Acid Bacteria, Use and Characterization of the Fermentation Process

The selective process includes the characterization of the two biotypes Lactobacillus brevis DSM 33325 and Lactobacillus plantarum DSM 33326 (deposited in the Deutsche Sammlung von Mikroorganismen and Zellkulturen GmbH culture collection by Celery, applicant of the present patent application) and a cluster of previous microorganisms identified in scientific research, as potential starters to be used in food biotechnologies, with the aim of highlighting the differences in pro-technological and nutritional performances considered of interest for the purposes of the application subject of the present patent application.

### Materials and Methods

### Strains of Microorganisms

Different microorganisms allocated to the Culture collection of the Department of Soil, Plant and Food Sciences
Di.S.S.P.A. (University of Ban, Italy), previously isolated from quinoa, hemp, wheat germ and chickpea, were used for the selection of starters useful for the production of a food composition according to the present invention, precisely:
Lactobacillus brevis DSM 33325 and Lactobacillus plantarum DSM 33326, originally isolated from plant matrices for food use and deposited in the Deutsche Sammlung von Mikroorganismen and Zellkulturen GmbH culture collection by Celery, applicant of the present patent application;
Lb. plantarum 18S9, Pediococcus acidilactici 10MMM0 and Leuconostoc mesenteroides 12MM1 (Nionelli et al., 2018. Pro-technological and functional characterization of lactic acid bacteria to be used as starters for hemp (Cannabis sativa L.) sourdough fermentation and wheat bread fortification. International Journal of Food Microbiology, 279:14-25);
Lb. plantarum LB1 and Lactobacillus rossiae LB5 (Rizzello et al., 2010. Effect of sourdough fermentation on stabilization, and chemical and nutritional characteristics of wheat germ. Food Chemistry, 119: 1079-1089);
Lb. plantarum MRS1, MR10, (Rizzello et al., 2014. Use of sourdough fermentation and mixture of wheat, chickpea, lentil and bean flours for enhancing the nutritional, texture and sensory characteristics of white bread. International Journal of Food Microbiology, 180:78-87).

### Fermentation of Vegetable Matrices, Test Conditions.

The strains of the microorganisms reported in the previous section were propagated in MRS (broth) at 30° C. for 24 hours. Cells were harvested by centrifugation (10,000 rpm, 10 min, 4° C.), washed twice in sterile potassium phosphate buffer (50 mM, pH 7.0), resuspended in water at the cell density of live and viable cells of about 10⁸ cfu/ml and used as a starter for the fermentation of suspensions of cereal, legume and pseudocereal flours (initial cell density of the dough, about 10⁷ cfu/g), with the aim of monitoring the main pro-technological characteristics. The test suspensions contained 10% (weight/volume) of flour in water. The flours used for screening were: chickpea, rice and quinoa. Fermentation was carried out in triple replicates at 30° C. for 24 hours.

### Fermentation Performance: Enumeration, pH, Synthesis of Lactic and Acetic Acids, Determination of the Concentration in Free Amino Acids

Strains of microorganisms were enumerated using MRS agar (Oxoid, Basingstoke, Hampshire, UK), with the addition of cycloheximide (0.1 g/l). The plates were incubated, in anaerobiosis (AnaeroGen and AnaeroJar, Oxoid), at 30° C. for 48 hours. The pH was measured using a pH meter (Model 507, Crison, Milan, Italy) provided with a probe for solid food. The aqueous extracts of the samples were prepared according to the protocol reported by Weiss et al. (1993) (Weiss W, Vogelmeier C, Gorg A. 1993. Electrophoretic characterization of wheat grain allergens from different cultivars involved in bakers' asthma. Electrophoresis, 14:805-816) and used for the determination of the concentration of organic acids (lactic acid and acetic acid), and of total free amino acids. Organic adds were determined by high performance liquid chromatography (HPLC), using an AKTA Purifier system (GE Healthcare, Buckinghamshire, UK) provided with an Aminex HPX-87H column (ion exclusion, Bio-Rad, Richmond, Calif., United States) and a 210 nm UV detector. The elution was carried out at 60° C., with a flow rate of 0.6 ml/min, using 10 mM H₂SO₄ as mobile phase (Rizzello et al., 2010. Effect of sourdough fermentation on stabilization, and chemical and nutritional characteristics of wheat germ. Food Chemistry, 119:1079-1089). The concentration of free amino adds was determined using a Biochrom series 30 amino add analyzer (Biochrom Ltd., Cambridge Science Park, UK) with a cation exchange column (inner diameter of 0.46 cm), by post column derivatization with ninhydrin, such as described by (Rizzello et al., 2010).

### Determination of the Degradation of Anti-Nutritional Compounds: Condensed Tannins, Raffinose, Phytic Acid, Saponins Present in Yoghurt-Like Food

Condensed tannins were determined using the butanoic acid test (Hagerman, 2002. Acid buthanol assay for proanthocyanidins A. E. Hagerman (Ed.), The Tannin Handbook, Miami University, Oxford). The concentrations of phytic acid and raffinose were determined using the K-PHYT 05/07 kit (Megazyme International, Ireland) and the Raffinose/Galactose kit (Megazyme International, Ireland), according to the manufacturer's instructions, respectively. In particular, to allow the analysis, raffinose was hydrolyzed to galactose and sucrose by the enzyme alpha-galactosidase. Total saponins were determined with the method previously proposed by Lai et al. (2013) with modifications related to the extraction phase (Lai et al., 2013. Effect of lactic fermentation on the total phenolic, saponin and phytic acid contents as well as anti-colon cancer cell proliferation activity of soymilk. Journal of Bioscience and Bioengineering, 115: 552-6). Briefly, the freeze-dried food (0.5 g) was mixed with 10 ml of petroleum ether under stirring for 4 hours. The residues (20 mg) were then extracted with 5 ml of aqueous methanol at 80% (vol/vol) under stirring for 4 hours. The extracts were kept at 4° C. in the dark until they were analyzed. The total saponin content (TSC) was determined using the spectrophotometric method proposed by Lai et al. (2013).

### Determination of the Ability to Increase the Concentration of Polyphenols and Antioxidant Activity of the Fermented Matrix.

Total polyphenols were determined on methanol extract. For the preparation of these extracts, 5 grams of each sample were mixed with 50 ml of 80% methanol. The mixture was degassed with nitrogen flow for 30 minutes, under stirring conditions, and centrifuged at 4600×g for 20 minutes. The extracts were transferred to falcon, degassed with nitrogen flow and stored at 4° C. before analysis. The total polyphenol concentration was determined and expressed as gallic add equivalents (Slinkard and Singleton, 1977. Total phenol analysis: automation and comparison with manual methods. American Journal of Enology and Viticulture, 28: 49). The radical DPPH. was used for the determination of the scavenging activity of methanolic extracts (Rizzello et al., 2010). Synthetic antioxidant butyl hydroxytoluene (BHT) (75 ppm) was included in the analysis as a positive reference: The reaction was monitored by determining the absorbance at 517 nm.

### Results

### Capacity for Growth, Acidification and Production of Free Amino Acids

Lactobacillus brevis DSM 33325, Lactobacillus plantarum DSM 33326, Lb. plantarum 18S9, Pediococcus acidilactici 10MMM0, Leuconostoc mesenteroides 12MM1, Lb. plantarum LB1, Lactobacillus rossiae LB5, and Lb. plantarum MRS1 and MR10 were inoculated individually in each of the three selected matrices or semi-liquid suspensions of chickpea, rice and quinoa flours. The selection of the strains was based on the growth and acidification capacities, paying attention to the strains that showed optimal performance and adaptability in the three matrices considered. Tab. 1 shows the specific data found, the range and the median of the distribution for each strain of microorganism tested.

As reported in Table 1, in all the matrices tested the Lactobacillus brevis DSM 33325 and Lactobacillus plantarum DSM 33326 strains showed the highest cell density at the end of incubation, having values of Δ Log cfu/ml significantly higher than the value shown from the other strains tested and with respect to the median value relative to the distribution of the values of all the strains tested. Specifically, L. brevis DSM 33325 showed Δ Log cfu/ml equal to 2.2±0.2 in chickpea, 2.0±0.1 in rice and 2.2±0.2 in quinoa; L. plantarum DSM 33326 has Δ Log cfu/ml equal to 2.0±0.1, 2.3±0.1 and 2.0±0.1, respectively in chickpea, rice and quinoa; the median is 1.7 in chickpea and quinoa, 1.6 in rice.

Furthermore, all the strains listed above were tested on the basis of pH acidification capacity (ΔpH). As reported in Table 1, after 24 hours of fermentation of the single matrices, the strains Lactobacillus brevis DSM 33325 and Lactobacillus plantarum DSM 33326 showed the highest values of ΔpH both with respect to the value shown by the other strains tested and with respect to the median value relative to the distribution of the values of all tested strains. Specifically, L. brevis DSM 33325 has ΔpH equal to 2.3±0.1 in chickpea, 2.1±0.1 in rice and 2.3±0.1 in quinoa; L. plantarum DSM 33326 has ΔpH equal to 2.5±0.1, 2.0±0.1 and 2.5±0.1, respectively in chickpea, rice and quinoa; the median is 1.6 in chickpea and quinoa, 1.5 in rice.

As shown in Table 1, in accordance with the decrease in pH, the concentration of lactic acid and acetic acid produced during fermentation was higher in all the matrices in the L. plantarum DSM 33326 strain both with respect to the value shown by the other strains tested and with respect to the median value relative to the distribution of the values of all the tested strains; the concentration of lactic acid produced during fermentation was higher in all matrices in the L. brevis DSM 33325 strain both with respect to the value shown by the other strains tested and with respect to the median value relating to the distribution of the values of all the strains tested; the concentration of acetic acid produced during fermentation was higher in the rice in the L. brevis DSM 33325 strain compared to the median value obtained from the analysis of all the strains considered.

As shown in Table 1, the Lactobacillus brevis DSM 33325 and Lactobacillus plantarum DSM 33326 strains showed the highest release values of total free amino acids (TFAA) during the fermentation process both with respect to the value shown by the other strains tested and to the median relative to the distribution of the values of all strains tested.

TABLE 1
Growth (Δlog cfu/ml), acidification (ΔpH), production of organic acids
(mmol/L) and release of free amino acids (TFAA mg/L) in fermented matrices
based on chickpea, rice and quinoa by each of the strains bacterial tested,
Range and Median. Each value was expressed as the mean ± standard deviation (n = 3).

| | | | | |
|---|---|---|---|---|
| | | Lactic acid | Acetic acid | TFAA |
| Δlog cfu/ml | ΔpH | mmol/L | mmol/L | mg/L |

Chickpea

| | | | | | |
|---|---|---|---|---|---|
| DSM 33325 | 2.2 ± 0.2 | 2.3 ± 0.1 | 10 ± 0.1 | 2.0 ± 0.1 | 200 ± 4 |
| DSM 33326 | 2,0 ± 0.1 | 2.5 ± 0.1 | 16 ± 0.4 | 5.0 ± 0.2 | 230 ± 3 |
| 18S9 | 1.6 ± 0.1 | 1.7 ± 0.1 | 6 ± 0.5 | 2 ± 0.1 | 155 ± 3 |
| 10MMM0 | 1.7 ± 0.1 | 1.7 ± 0.1 | 7 ± 1.0 | 3 ± 0.1 | 160 ± 3 |
| 12MM1 | 1.7 ± 0.1 | 1.5 ± 0.1 | 7 ± 0.5 | 2 ± 0.2 | 135 ± 2 |
| LB1 | 1.6 ± 0.2 | 1.5 ± 0.1 | 6 ± 0.5 | 3 ± 0.1 | 130 ± 3 |
| LB5 | 1.7 ± 0.1 | 1.5 ± 0.1 | 6 ± 0.5 | 4 ± 0.3 | 130 ± 3 |
| MRS1 | 1.5 ± 0.1 | 1.3 ± 0.1 | 8 ± 0.5 | 2 ± 0.2 | 110 ± 2 |
| MR10 | 1.6 ± 0.2 | 1.6 ± 0.2 | 5 ± 0.5 | 3 ± 0.1 | 143 ± 1 |
| Range | 1.5-2.2 | 1.3-2.5 | 5.0-16.0 | 2.0-5.0 | 110-230 |
| Median | 1.7 | 1.6 | 7.0 | 3.0 | 143 |

Rice

| | | | | | |
|---|---|---|---|---|---|
| DSM 33325 | 2.0 ± 0.1 | 2.1 ± 0.1 | 9.0 ± 0.2 | 3.0 ± 0.1 | 70 ± 1 |
| DSM 33326 | 2.3 ± 0.1 | 2.0 ± 0.1 | 13.0 ± 0.4 | 3.0 ± 0.1 | 50 ± 2 |
| 18S9 | 1.5 ± 0.1 | 1.6 ± 0.1 | 7 ± 0.1 | 2 ± 0.1 | 38 ± 2 |
| 10MMM0 | 1.7 ± 0.1 | 1.4 ± 0.1 | 6 ± 0.1 | 2 ± 0.2 | 42 ± 2 |
| 12MM1 | 1.6 ± 0.2 | 1.5 ± 0.1 | 8 ± 0.1 | 2 ± 0.1 | 37 ± 2 |
| LB1 | 1.5 ± 0.1 | 1.6 ± 0.1 | 6 ± 0.1 | 3 ± 0.3 | 42 ± 2 |
| LB5 | 1.6 ± 0.1 | 1.5 ± 0.1 | 6 ± 0.3 | 2 ± 0.1 | 40 ± 2 |
| MRS1 | 1.6 ± 0.1 | 1.4 ± 0.1 | 8 ± 0.2 | 2 ± 0.1 | 34 ± 2 |
| MR10 | 1.5 ± 0.2 | 1.4 ± 0.1 | 5 ± 0.5 | 3 ± 0.2 | 36 ± 2 |
| Range | 1.5-2.3 | 1.4-2.1 | 5.0-13.0 | 2.0-3.0 | 34-70 |
| Median | 1.6 | 1.5 | 7.0 | 2.0 | 40 |

Quinoa

| | | | | | |
|---|---|---|---|---|---|
| DSM 33325 | 2.2 ± 0.2 | 2.3 ± 0.1 | 10 ± 0.3 | 2 ± 0.1 | 200 ± 6 |
| DSM 33326 | 2.0 ± 0.1 | 2.5 ± 0.1 | 16 ± 0.3 | 5 ± 0.2 | 230 ± 7 |
| 18S9 | 1.7 ± 0.1 | 1.8 ± 0.1 | 6 ± 0.2 | 2 ± 0.1 | 150 ± 3 |
| 10MMM0 | 1.7 ± 0.1 | 1.7 ± 0.1 | 7 ± 0.5 | 3 ± 0.2 | 155 ± 2 |
| 12MM1 | 1.5 ± 0.2 | 1.6 ± 0.2 | 7 ± 0.5 | 2 ± 0.2 | 143 ± 3 |
| LB1 | 1.5 ± 0.1 | 1.4 ± 0.1 | 6 ± 0.2 | 3 ± 0.2 | 130 ± 4 |
| LB5 | 1.7 ± 0.1 | 1.4 ± 0.1 | 7 ± 0.5 | 4 ± 0.2 | 120 ± 2 |
| MRS1 | 1.5 ± 0.1 | 1.3 ± 0.1 | 7 ± 0.5 | 2 ± 0.2 | 110 ± 3 |
| MR10 | 1.8 ± 0.1 | 1.4 ± 0.2 | 5 ± 0.3 | 3 ± 0.2 | 120 ± 2 |
| Range | 1.5-2.2 | 1.3-2.5 | 5.0-16.0 | 2.0-5.0 | 110-230 |
| Median | 1.7 | 1.6 | 7.0 | 3.0 | 143 |

### Degradative Capacity of Anti-Nutritional Factors

The ability to reduce the concentration of anti-nutritional factors present in cereals, legumes and pseudocereals was evaluated; such factors, such as for example tannins, raffinose, phytic acid and saponins can in fact contribute negatively to the digestibility of the food. The potential possibility of reducing the concentration of these factors through fermentation processes has been reported in the literature (Gobbetti et al., 2014; Gobbetti et al., 2019a; Gobbetti et al., 2019b. The sourdough fermentation is the powerful process to exploit the potential of legumes, pseudo-cereals and milling by-products in baking industry. Critical reviews in food science and nutrition, 1-16). Tab. 2 shows the specific data found, the range and the median of the distribution for each strain of microorganism tested.

As shown in Table 2, the concentration of tannins, raffinose, phytic acid and saponins in all the matrices fermented with the strains Lactobacillus brevis DSM 33325 and Lactobacillus plantarum DSM 33326 is lower than both the concentration found in the corresponding matrices fermented with the other tested strains, and with respect to the median value relating to the distribution of the values of all tested strains.

TABLE 2
Effect of fermentation on the concentration of anti-nutritional
factors: concentration of tannins, raffinose, phytic acid and
saponins in fermented matrices based on chickpea, rice and quinoa
by each of the bacterial strains tested, Range and Median. Each
value was expressed as the mean ± standard deviation (n = 3).

| | | | |
|---|---|---|---|
| Tannins | Raffinose | Phytic acid | Saponins |
| mg/100 ml | mg/100 ml | mg/100 ml | mg/100 ml |

Chickpea

| | | | | |
|---|---|---|---|---|
| DSM 33325 | 1.51 ± 0.10 | 10 ± 1 | 40.5 ± 1.1 | 31.1 ± 0.7 |
| DSM 33326 | 1.52 ± 0.30 | 12 ± 2 | 40.0 ± 1.2 | 30.2 ± 0.8 |
| 18S9 | 2.10 ± 0.05 | 18 ± 1 | 50.5 ± 1.0 | 50.2 ± 1.0 |
| 10MMM0 | 2.04 ± 0.10 | 20 ± 1 | 50.0 ± 1.1 | 60.5 ± 1.5 |
| 12MM1 | 2.00 ± 0.15 | 20 ± 1 | 66.0 ± 1.5 | 50.5 ± 1.0 |
| LB1 | 2.40 ± 0.10 | 22 ± 1 | 70.5 ± 0.5 | 60.0 ± 1.0 |
| LB5 | 2.05 ± 0.10 | 10 ± 2 | 70.0 ± 1.5 | 65.2 ± 1.5 |
| MRS1 | 3.02 ± 0.15 | 18 ± 1 | 73.5 ± 0.5 | 50.1 ± 1.0 |
| MR10 | 2.00 ± 0.05 | 17 ± 1 | 70.5 ± 1.3 | 60.2 ± 0.5 |
| Range | 1.51-3.02 | 10-22 | 40.0-73.5 | 31.1-65.2 |
| Median | 2.05 | 18 | 65.0 | 50.5 |

Rice

| | | | | |
|---|---|---|---|---|
| DSM 33325 | 0.52 ± 0.02 | 5 ± 1 | 32.3 ± 0.9 | N/D |
| DSM 33326 | 0.82 ± 0.03 | 6 ± 1 | 21.5 ± 0.8 | N/D |
| 18S9 | 0.92 ± 0.02 | 10 ± 1 | 55.2 ± 0.3 | N/D |
| 10MMM0 | 1.32 ± 0.01 | 11 ± 2 | 54.3 ± 0.2 | N/D |
| 12MM1 | 1.04 ± 0.01 | 10 ± 2 | 65.2 ± 0.5 | N/D |
| LB1 | 1.56 ± 0.02 | 10 ± 1 | 60 ± 0.2 | N/D |
| LB5 | 1.03 ± 0.02 | 9 ± 1 | 62.9 ± 0.8 | N/D |
| MRS1 | 1.12 ± 0.01 | 8 ± 1 | 60.3 ± 0.2 | N/D |
| MR10 | 1.23 ± 0.03 | 9 ± 2 | 73 ± 0.5 | N/D |
| Range | 0.52-1.56 | 5-11 | 21.5-73.0 | N/D |
| Median | 1.04 | 9 | 60.0 | N/D |

Quinoa

| | | | | |
|---|---|---|---|---|
| DSM 33325 | 1.50 ± 0.13 | 10 ± 2 | 41.5 ± 0.1 | 50.5 ± 1.4 |
| DSM 33326 | 1.52 ± 0.12 | 12 ± 1 | 41.0 ± 0.1 | 50.0 ± 0.7 |
| 18S9 | 2.22 ± 0.10 | 18 ± 1 | 55.0 ± 0.1 | 86.0 ± 1.0 |
| 10MMM0 | 2.8 ± 0.05 | 20 ± 2 | 50.0 ± 0.2 | 90.0 ± 0.5 |
| 12MM1 | 2.64 ± 0.10 | 20 ± 1 | 64.3 ± 0.1 | 80.8 ± 0.3 |
| LB1 | 3 ± 0.08 | 23 ± 2 | 75.6 ± 0.2 | 86.0 ± 0.1 |
| LB5 | 2.1 ± 0.10 | 18 ± 1 | 72.0 ± 0.1 | 93.0 ± 0.3 |
| MRS1 | 2.52 ± 0.15 | 20 ± 2 | 73.3 ± 0.2 | 90.5 ± 0.2 |
| MR10 | 2.34 ± 0.05 | 17 ± 1 | 70.2 ± 0.5 | 90.4 ± 0.6 |
| Range | 1.50-3.20 | 10-23 | 41.0-75.6 | 50.0-93.0 |
| Median | 2.34 | 18 | 64.3 | 86.0 |

N/D: not determinable

### Polyphenols and Antioxidant Activity

The ability to modify the concentration of polyphenols in the fermented matrix and consequently the respective antioxidant activity was evaluated; in fact, through acidification and specific metabolic activities, lactic acid bacteria are able to lead to a greater solubilization of polyphenols and to the release of simple molecules starting from complex and glycosylated forms (Gobbetti et al., 2019b). This results in a greater bioavailability of these compounds for the human body and a more marked antioxidant capacity (anti-age) in vivo. The antioxidant activity was determined by studying the scavenging (detoxifying) activity on the synthetic radical DPPH. (Rizzello et al., 2010). Tab. 3 shows the specific data found, the range and the median of the distribution for each strain of microorganism tested.

As shown in Table 3, the concentration of polyphenols in all the matrices fermented with the strains Lactobacillus brevis DSM 33325 and Lactobacillus plantarum DSM 33326 is greater than both the concentration found in the corresponding matrices fermented with the other tested strains, and with respect to the median value relating to the distribution of the values of all tested strains. To confirm the data, the antioxidant activity of the matrices fermented with Lactobacillus brevis DSM 33325 and Lactobacillus plantarum DSM 33326 was higher than of those fermented with the other strains tested.

TABLE 3
Effect of fermentation on the concentration in total phenols
and on the antioxidant activity of fermented matrices
based on chickpea, rice and quinoa by each of the bacterial
strains tested, Range and Median. Each value was expressed
as the mean ± standard deviation (n = 3).

| | |
|---|---|
| | Antioxidant activity |
| Polyphenols | (radical scavenging |
| mmol/L | activity on DPPH) % |

Chickpea

| | | |
|---|---|---|
| DSM 33325 | 0.100 ± 0.010 | 45.0 ± 0.5 |
| DSM 33326 | 0.100 ± 0.010 | 50.0 ± 0.6 |
| 18S9 | 0.080 ± 0.005 | 34.0 ± 1.0 |
| 10MMM0 | 0.090 ± 0.005 | 32.0 ± 0.5 |
| 12MM1 | 0.080 ± 0.010 | 35.0 ± 0.5 |
| LB1 | 0.080 ± 0.005 | 36.0 ± 1.0 |
| LB5 | 0.070 ± 0.005 | 35.0 ± 0.5 |
| MRS1 | 0.070 ± 0.005 | 33.0 ± 0.5 |
| MR10 | 0.070 ± 0.005 | 35.0 ± 0.5 |
| Range | 0.070-0.100 | 32.0-50.0 |
| Median | 0.080 | 35.0 |

Rice

| | | |
|---|---|---|
| DSM 33325 | 0.090 ± 0.006 | 30.0 ± 0.2 |
| DSM 33326 | 0.070 ± 0.002 | 34.0 ± 0.1 |
| 18S9 | 0.010 ± 0.001 | 10.0 ± 0.2 |
| 10MMM0 | 0.001 ± 0.001 | 12.0 ± 0.2 |
| 12MM1 | 0.002 ± 0.001 | 13.0 ± 0.2 |
| LB1 | 0.001 ± 0.001 | 12.0 ± 0.2 |
| LB5 | 0.002 ± 0.001 | 12.0 ± 0.3 |
| MRS1 | 0.001 ± 0.001 | 12.0 ± 0.3 |
| MR10 | 0.002 ± 0.001 | 12.0 ± 0.2 |
| Range | 0.001-0.090 | 10.0-34.0 |
| Median | 0.002 | 13.0 |

Quinoa

| | | |
|---|---|---|
| DSM 33325 | 0.200 ± 0.010 | 55.0 ± 0.9 |
| DSM 33326 | 0.300 ± 0.010 | 60.0 ± 0.8 |
| 18S9 | 0.080 ± 0.005 | 33.0 ± 0.5 |
| 10MMM0 | 0.070 ± 0.005 | 32.0 ± 0.5 |
| 12MM1 | 0.080 ± 0.005 | 34.0 ± 0.9 |
| LB1 | 0.090 ± 0.005 | 32.0 ± 0.5 |
| LB5 | 0.090 ± 0.005 | 34.0 ± 0.3 |
| MRS1 | 0.090 ± 0.005 | 33.0 ± 1.0 |
| MR10 | 0.070 ± 0.005 | 33.0 ± 0.5 |
| Range | 0.070-0.300 | 32.0-60.0 |
| Median | 0.090 | 35.0 |

Based on the results shown in Tab. 1-3, it can be seen that the L. brevis DSM 33325 and L. plantarum DSM 33326 strains showed the highest cell density at the end of the incubation; higher values of ΔpH due to the increased production of lactic acid and acetic acid during fermentation; higher release values of free total amino acids; greater ability to reduce the concentration of anti-nutritional factors present in cereals, legumes and pseudocereals which can negatively contribute to the digestibility of food; greater ability to make polyphenols bioavailable for the human body which determines a more marked antioxidant capacity (anti-aging) in vivo. These distinctive capabilities and performances make the L. brevis DSM 33325 and L. plantarum DSM 33326 strains suitable for being selected to be used as a starter in the production of a food composition according to the invention.

### Example 2: Production of a Yoghurt-Like Food Composition Base Formulation (BYL-Base) Representative of the Invention and an Unfermented Control Composition (cBYL)

### Materials and Methods

### Microorganisms and Cultivation Conditions

L. brevis DSM 33325 and L. plantarum DSM 33326 were grown in MRS broth at 30° C. for 24 hours until the late exponential growth phase (approx. 8 hours) was reached, as confirmed by the growth kinetic analysis (Pontonio et al., 2015. Iranian wheat flours from rural and industrial mills: Exploitation of the chemical and technology features, and selection of autochthonous sourdough starters for making breads. Food Microbiology 47:99-110).

The cells were recovered by centrifugation (10,000 rpm, 10 min, 4° C.), washed twice in phosphate buffer (50 mM and pH 7.0) and then resuspended in drinking water at a cell density of live and viable cells of approx. 10⁷ cfu/ml.

### Nutritional Label of Raw Materials

Rice, chickpea and lentil flours were used, chosen on the basis of complementary nutritional properties and on the basis of technological properties. The rice flour was selected on the basis of its good rheological and organoleptic properties. Preliminary measurements of the viscosity of the matrix were carried out in order to determine the percentage of rice flour and legume flours to be used in the final product. Proteins (N×5.7), lipids, moisture, total dietary fiber content and ash of the raw materials used were determined according to approved methods 46-11A, 30-10.01, 44-15A, 32-05.01 and 08-01.01 by the American Association of Cereal Chemists (MCC. 2010. Approved methods of analysis. St. Paul: Approved Methods Committee. Available from: http://methods.aaccnet.org/. Accessed 2015 Dec. 18). Carbohydrates were calculated as the difference [100−(protein+fat+ash+total dietary fiber)]. Proteins, lipids, carbohydrates, total dietary fiber and ash were expressed as % dry matter (s.s.).

### Production

Rice flour (gluten-free cereal flour, A), chickpea and lentil (legume flours, B) in a 2:1:1 ratio (w/w/w) were mixed with water (80% vol/w).

The suspension, suitably mixed, was heat treated at 80° C. for 15 min.

After a sudden cooling of the gelatinized mixture to 4° C., the mixture was conditioned at 30° C. and inoculated with a cell suspension of L. brevis DSM 33325 and L. plantarum DSM 33326 in a 1:1 cell density ratio in order to obtain a cell density of live and viable cells of about 2×10⁷ cfu/ml. Fermentation was carried out at 30° C. for 12 hours. At the end of the incubation, the mixture was cooled to 4° C. thus obtaining the yoghurt-like base (BYL-base) food composition representative of the invention:

Under the same technological conditions reported above, a control yoghurt-like food composition (cBYL) was produced in which the matrix consisting of rice flour, chickpea and lentil in a ratio of 2:1:1 (w/w/w) was mixed with water (80% vol/w) but was not fermented, i.e. it was not inoculated with the L. brevis DSM 33325 and L. plantarum DSM 33326 bacteria starter in a 1:1 cell density ratio.

### Description of the Tests—pH and Enumeration, Kinetics of Growth and Acidification, Determination of Titratable Acidity, Viscosity And Concentration of Organic Acids

The yoghurt-like base food composition (BYL-base) and the control composition (cBYL) prepared as described above were analyzed within 2 hours from the end of fermentation.

Enumeration of lactic acid bacteria was performed using MRS (Oxoid, Basingstoke, Hampshire, UK) agar, containing with cycloheximide (0.1 g/I) as substrate. The growth and acidification kinetics were determined and modeled according to the Gompertz equation, modified by Zwietering et al. (1990):

y=k+A exp{−exp[(μmax or V max·and/A)(λ−t)+1]}

wherein
y is the growth expressed as log cfu/g/h or the acidification expressed as dpH/dt (units of pH/h) at time t; k is the initial level of the dependent variable (log cfu/g or pH unit);
A is the change in cell density or pH between the inoculum and the stationary phase;
μmax or Fmax are the maximum growth rate expressed as Δlog cfu/g/h and the maximum acidification rate expressed as dpH/h, respectively;
λ is the duration of the latency phase measured in hours.

The kinetics were obtained by extending the fermentation to 18 hours, to obtain a Gompertz modeling that adequately described the growth and acidification phases (lag, exponential and stationary).

The experimental data were processed with a non-linear regression with the Statistica 8.0 software (Statsoft, Tulsa, USA).

The pH was measured using a pH meter (Model 507, Crison, Milan, Italy) provided with a probe for solid food. Titration acidity (TTA) was determined on 10 g of food composition homogenized in 90 ml of distilled water for 3 minutes in a Mixer 400P bag (interscience, St Nom, France), and expressed as the quantity (ml) of 1 M NaOH used to reach a pH value of 8.3.

Apparent viscosity was measured on approximately 35 ml of food composition, using the A & D SV-10 sine wave vibro-viscometer (A & D Company Ltd., Japan). Viscosity measurements were performed on beverages previously kept at 25° C. for 30 minutes.

The aqueous extracts were prepared as specified in Example 1 and used for the determination of the concentration of organic acids (lactic acid and acetic acid).

### Results

### Nutritional Characteristics of Flour

Table 4 shows the nutritional label (on 100 g of product) of the rice, chickpea and lentil flours used as fermentation matrices in the present invention. The energy value of these flours is 350, 291 and 363 Kcal, respectively. Among the matrices considered, the highest fiber intake corresponds to lentils and chickpeas, 8.10±0.40% and 11.10±0.30%, respectively, while for rice it is 3.20±0.60%. The protein content is also higher for chickpea (22.00±0.70%) and lentils (25.80±0.60%), while it is equal to 8±0.6% in rice flour.

TABLE 4
Nutritional label of rice, chickpea and lentil flours, used as
matrices for the production of the yoghurt-like base (BYL-base)
food composition. Data are expressed as % of dry matter. Each
value was expressed as the mean ± standard deviation (n = 3).

| | Rice | Chickpea | Lentil |
|---|---|---|---|
| Energy value | 350 Kcal | 363 Kcal | 291 Kcal |
| Fat | 2.50 ± 0.10 | 5.00 ± 0.10 | 1.80 ± 0.04 |
| of which saturated | 0.50 ± 0.02 | 0.60 ± 0.03 | 0.16 ± 0.02 |
| fatty acids | | | |
| Carbohydrates | 72.00 ± 1.50 | 52.00 ± 1.50 | 59.40 ± 1.50 |
| Fibers | 3.20 ± 0.60 | 11.10 ± 0.30 | 8.10 ± 0.40 |
| Proteins | 8.20 ± 0.60 | 22.00 ± 0.70 | 25.80 ± 0.60 |
| Ashes | 0.61 ± 0.02 | 2.86 ± 0.20 | 3.7 ± 0.10 |

Growth Curves and Acidification by the Selected Starter (L. brevis DSM 33325 and L. plantarum DSM 33326) in the Yoghurt-Like Base Food Composition (BYL-Base).

Growth and acidification curves were obtained by Gompertz modeling.

During the fermentation process monitored for a duration of 18 hours of incubation at 30° C., the cell density of the starter increased, by approximately 2 logarithmic cycles, from 7.41±0.16 log cfu/mL to 9.43±0.36 log cfu/mL in BYL.

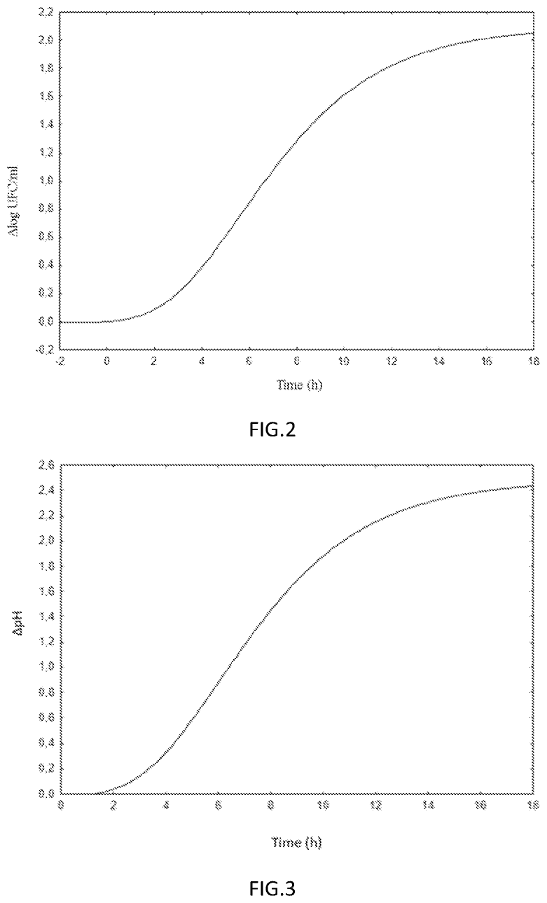

As shown in FIG. 2, the relative Δlog cfu/mL is 2.01. The latency phase (A) was equal to 2.44 hours, the Vₘₐₓ value was 0.23; in the cBYL control sample, lactic acid bacteria are completely absent.

During the fermentation process, monitored for a duration of 18 hours of incubation at 30° C., the initial pH value was 6.50±0.18 and changed to 4.04±0.21 after fermentation.

As shown in FIG. 3, the relative ΔpH is 2.48. The values of Vₘₐₓ and λ relative to the acidification kinetics were 0.30 and 3.04, respectively.

In the control sample, however, no significant reduction in pH was observed, as the initial value was equal to 6.50±0.16 and the final value is 5.97±0.23, with a relative ΔpH equal to 0.53.

Physical-Chemical Analysis of Organic Acids in the Base Yoghurt-Like Food Composition (BYL-Base) and in the Control Composition (cBYL).

As shown in Table 5, the apparent viscosity value of BYL-base amounted to 4.23±0.11 Pa·s, which is significantly greater than the control sample cBYL where the value is 3.7±0.13 Pa·s; the titratable acidity value is significantly greater in BYL-base where the value is 5.80±0.15 mL of 0.1 M NaOH per 100 g of product with respect to the control sample cBYL where the value is 2.2±0.09; in the presence of the mixed starter L. brevis DSM 33325 and L. plantarum DSM 33326, fermentation leads to the synthesis of lactic acid (11.9±0.2 mmol/L) and acetic acid (5.4±0.2 mmol/L), not found in cBYL.

TABLE 5
Viscosity, titration acidity (TTA) and concentration of organic
acids in the BYL-base composition in comparison with a cBYL
control composition produced under the same technological conditions,
but without the inoculation of selected starter. Each value
was expressed as the mean ± standard deviation (n = 3).

| | | TTA | Lactic | Acetic |
|---|---|---|---|---|
| | Viscosity | (ml NaOH | acid | acid |
| Samples | (Pa · s) | 0.1M/100 g) | (mmol/L) | (mmol/L) |
| cBYL | 3.70 ± 0.13 | 2.20 ± 0.09 | n/d. | n/d |
| BYL-base | 4.23 ± 0.11 | 5.80 ± 0.15 | 11.9 ± 0.2 | 5.4 ± 0.2 |

### Example 3: Nutritional and Functional Characteristics in the Base Yoghurt-Like Food Composition (BYL-Base) and in the Control Composition (cBYL) Unfermented

### Materials and Methods

The base yoghurt-like food composition (BYL-base) and the cBYL control were prepared as described in Example 2.

### Nutritional Label

Protein (N×5.7), lipids, moisture, total dietary fiber and ash of the yoghurt-like food composition base (BYL-base) representative of the invention and of the control composition (cBYL) were determined according to the approved methods 46-11A, 30-10.01, 44-15A, 32-05.01 and 08-01.01 by the American Association of Cereal Chemists (2010). Carbohydrates were calculated as the difference [100−(protein+fat+ash+total dietary fiber)]. Proteins, lipids, carbohydrates, total dietary fiber and ash were expressed as % dry matter (d.m.).

### Concentration in Free Amino Acids

The concentration of free amino acids was determined by post-column derivatization by a Biochrom 30 analyzer, as described in Example 1.

### Determination of the In Vitro Digestibility of Proteins

in vitro protein digestibility (IVPD) was determined by the Akeson and Stahmann method (Akeson and Stahmann, 1964. A pepsin pancreatin digest index of protein quality evaluation, Journal of Nutrition, 83:257-261). One gram of sample was incubated with 1.5 mg of pepsin, in 15 ml of HCl, 0.1 M at 37° C. for 3 hours. After neutralization with 2M NaOH and addition of 4 mg of pancreatin, in 7.5 ml of phosphate buffer (pH 8.0), 1 ml of toluene was added to prevent microbial growth by incubating the solution for 24 hours at 37° C. After 24 hours, the enzyme was inactivated by adding 10 ml of trichloroacetic acid (20%, wt/vol) precipitating the undigested proteins. The volume was brought to 100 ml with distilled water and the solution centrifuged at 5000 g/min for 20 minutes. The protein concentration of the supernatant was determined by the Bradford method (Bradford, 1976. A rapid and sensitive method for the quantitation of microgram quantities of protein utilizing the principle of protein-dye binding. Analytical Biochemistry, 72:248-54). The precipitate was subjected to protein extraction (Weiss et al., 1993) and the protein concentration was determined. Protein digestibility in vitro was expressed as a percentage of total proteins, which were solubilized after enzymatic hydrolysis.

### Determination of Nutritional Indices

The calculation of the nutritional indices following the complete hydrolysis of the digestible fraction of the proteins (obtained for the determination of the IVPD as previously described) was carried out as described by Rizzello et al. (2014) and included the calculation of the Chemical Score, the essential amino acid index (EAAI), the biological value (BV), the protein efficiency ratio (PER) and the nutritional index (NI) which normalizes the qualitative and quantitative variations of the test protein with respect to nutritional status.

In particular, the free amino acids were determined by means of a Biochrom 30 automatic analyzer after complete acid hydrolysis of the digestible protein fraction. Since the procedure described does not allow the determination of tryptophan, it was determined by the Pinter-Szakács and Molnán-Perl method (1990) (Molnar-Perl et al., 1990. Gas chromatographic determination of isocitric and malic acid in the presence of a large excess of citric acid. Analytica Chimica Acta. 239: 165-170).

The data obtained were used for the calculation of the nutritional indices. The Chemical Score (CS) value, corresponding to the amount of protein required to provide the minimum pattern of essential amino acids (Essential Aminoacids, EM), was calculated with the Block and Mitchel equation (Block and Mitchel, 1946. The correlation of the amino-acid composition of protein with their nutritive value. Nutrition abstracts and reviews, 16: 249-278), which compares essential amino acids to that of the reference protein (egg). These data were used to obtain the list of limiting essential amino acids (having the lowest CS values) (Block and Mitchel, 1946). The protein score indicates the CS of the most limiting amino acid present in the sample protein (Block and Mitchel, 1946). The Essential Amino Acid Index (EAAI) estimates the quality of the test protein. It is calculated according to the Oser procedure (Oser, 1959. Protein and amino acid nutrition Albanese Academic Press, New York, 281-291), considering the ratio between EAA of the test protein and those of the reference protein, according to the formula:

$$EAAI = \sqrt[n]{\frac{( E A {A}_{1} * 100 ) ( E A {A}_{2} * 100 ) ( … ) ( E A {A}_{n} * 10 \; 0 ) [ sample ]}{( E A {A}_{1} * 100 ) ( E A {A}_{2} * 100 ) ( … ) ( E A {A}_{n} * 100 ) [ reference ]}}$$

The biological value (BV) indicates the usable fraction of the protein and is calculated according to the equation proposed by Oser (1959): BV=([1.09*EAAI]−11.70). The Protein Efficiency Ratio (PER) value estimates the nutritional quality of the protein based on the amino acid profile obtained by hydrolysis of the same; it was calculated according to the model developed by Ihekoronye (Ihekoronye, 1981. A rapid enzymatic and chromatographic predictive model for the in-vivo rat-based protein efficiency ratio, Ph.D. Thesis, University of Missouri, Columbia): PER=−0.468+(0.454*[Leucine])−(0.105*[Tyrosine]). Finally, the Nutritional Index (NI) normalizes the qualitative and quantitative variations of the protein, taking into account the nutritional potential; it is calculated with the Crisan and Sands equation (1978) (Crisan and Sands, 1978. Biology and cultivation of edible mushrooms, Academic Press, New York, 137-142), which considers all factors with equal importance: NI=(EAAI*Protein (%)/100).

### Determination of the Starch Hydrolysis Index and the Predicted Glycemic Index

The analysis of starch hydrolysis was carried out using a well-established laboratory procedure that mimics the conditions of in vivo digestion of starch (De Angelis et al., 2009. Sourdough fermentation as a tool for the manufacture of low-glyceric index white wheat bread enriched in dietary fiber, European Food Research and Technology, 229: 593-601). Aliquots of food composition, containing 1 g of starch, were subjected to sequential enzymatic digestion and the released glucose content was measured with D-Fructose/D-glucose assay kit (Megazyme). The degree of starch digestion was expressed as a percentage of potentially hydrolyzed starch after 180 minutes. The reference food used to estimate the hydrolysis index (HI=100) was wheat flour bread leavened with brewer's yeast. The estimate of the glycemic index (predicted GI) was carried out using the equation: GI=0.549×HI+39.71 (Capriles and Areas, 2013. Effects of prebiotic inulin-type fructans on structure, quality, sensory acceptance and glycemic response of gluten-free breads, Food Function, 4:104-110).

### Concentration in Polyphenols and Determination of Antioxidant Activity.

The concentration in total polyphenols and the antioxidant activity were determined on the methanol extract as previously described in Example 1.

### Determination of the Concentration in Anti-Nutritional Factors: Phytic Acid, Raffinose, Condensed Tannins, Saponins

The concentration of the anti-nutritional factors such as phytic acid, condensed tannins, raffinose and saponins was determined as previously described in Example 1.

### Results

Nutritional and Functional Characterization The nutritional label of base yoghurt-like food composition (BYL-base) and the control composition (cBYL) are shown in Table 6 (data expressed on 100 g of product). Specifically, the energy value of (BYL-base) was equal to 67.7 kcal per 100 g of product. The (BYL-base) contains fat at 0.59±0.09%. According to Regulation (EC) no. 1924/2006 of the European Parliament and of the Council of 20 Dec. 2006, relating to nutrition and health claims made on food products, the product can be defined as low-fat if it does not contain more than 3 g of fat per 100 g of product. Based on this regulation, the (BYL-base) may be defined as low-fat. The fiber content is equal to 4.10±0.20%; corresponding to 6 g of fiber per 100 kcal of equivalent product. According to the aforementioned Regulation, a food can be defined as having a high fiber content for the consumer only if it contains at least 3 g of fiber per 100 Kcal of equivalent product. The BYL-base may be defined with a high fiber content. In addition, the (BYL-base) is characterized by concentrations of protein equal to 3.24±0.16%, which cover more than 20% of the energy value of the product. According to the aforementioned regulation, this characteristic allows defining the food as “high in protein”. Therefore, (BYL-base) may be defined with a high protein content even in its basic formulation.

TABLE 6
Nutrition label of base yoghurt-like food
composition (BYL-base). Each value was expressed
as the mean ± standard deviation (n = 3).

| | 100 g |
|---|---|
| Energy value (Kcal) | 67.7 |
| Fat (g) | 0.59 ± 0.09 |
| Carbohydrates (g) | 12.75 ± 0.12 |
| Fiber (g) | 4.10 ± 0.20 |
| Protein (g) | 3.24 ± 0.16 |
| Ashes (g) | 0.39 ± 0.02 |
| Moisture (g) | 79.27 ± 2.45 |

### Concentration in Free Amino Acids and GABA

Among the various advantages of fermentation by lactic acid bacteria is a proteolysis process that involves an increase in the peptide and/or amino acid content (Coda et al., 2014. Sourdough lactic acid bacteria: exploration of non-wheat cereal-based fermentation. Food Microbiology, 37:51-58; Nionelli et al., 2014. Manufacture and characterization of a yogurt-like beverage made with oat flakes fermented by selected lactic acid bacteria, International journal of food microbiology, 185C: 17-26). The (BYL-base) has a 1181.9 mg/L content of total free amino acids (TFAA), while in cBYL it is equal to 717.6 mg/L (Table 7). As previously demonstrated, the fermentation carried out with lactic acid bacteria leads, thanks to the effective proteolysis carried out by the latter on native proteins, to the release of large quantities of free amino acids, increasing their bioavailability (Coda et al., 2014; Nionelli et al., 2014)

TABLE 7
Concentration (mg/L) in free amino acids in the control
composition (cBYL) and in the yoghurt-like base
(BYL-base) food composition. Each value was expressed
as the mean ± standard deviation (n = 3).

| Amino acid (mg/L) | cBYL | BYL-base |
|---|---|---|
| Cysteic acid | 4.3 ± 0.3 | 18.9 ± 0.8 |
| Aspartic acid | 40.2 ± 1.8 | 60.0 ± 3.0 |
| Threonine | 10.4 ± 0.2 | 23.3 ± 1.2 |
| Serine | 20.8 ± 0.8 | 32.4 ± 1.4 |
| Glutamic acid | 65.5 ± 1.0 | 93.5 ± 1.8 |
| Glycine | 13.0 ± 0.5 | 28.1 ± 1.4 |
| Alanine | 45.4 ± 1.2 | 60.1 ± 1.4 |
| Cysteine | 76.3 ± 0.4 | 142.4 ± 0.6 |
| Valine | 28.3 ± 0.6 | 44.5 ± 1.5 |
| Methionine | 10.6 ± 0.0 | 24.0 ± 0.1 |
| Isoleucine | 15.7 ± 0.1 | 28.3 ± 0.2 |
| Leucine | 30.0 ± 0.2 | 44.0 ± 0.5 |
| Tyrosine | 15.1 ± 0.4 | 29.5 ± 1.4 |
| Phenylalanine | 28.8 ± 0.3 | 43.5 ± 0.6 |
| GABA γ-aminobutyric acid | 90.9 ± 0.5 | 110.9 ± 0.6 |
| Histidine | 21.9 ± 0.4 | 34.7 ± 0.5 |
| Tryptophan | 38.4 ± 1.6 | 75.4 ± 1.1 |
| Ornithine | 35.9 ± 0.1 | 50.5 ± 4.3 |
| Lysine | 22.6 ± 0.5 | 40.0 ± 0.5 |
| Arginine | 65.5 ± 1.2 | 130.0 ± 0.4 |
| Proline | 37.9 ± 1.3 | 67.9 ± 1.6 |
| Total (TFAA) | 717.6 | 1181.9 |

### Digestibility of Proteins and Nutritional Indices

In order to mimic the in vivo digestion of proteins, a protocol was used that allowed to estimate the in vitro digestibility of the same (IVDP, In Vitro Digestibility Protein). The IVDP value in the cBYL control was equal to 67.34%, while the value increased to 79.53% in the BYL.

The nutritional indices, aimed at estimating the quality of the protein fraction, were determined on the digestible fraction of the protein which, as estimated by calculating the IVPD, changes significantly as a result of the fermentation process (thanks to the influence that lactic acid bacteria have on native proteins, by proteolysis, and on the degradation of anti-nutritional factors) (Gobbetti et al., 2019b). This allows evaluating the nutritional indices of the protein fraction that actually contributes to the nutritional state of the human body. The chemical scores on which the calculations of the nutritional indices are based refer to the composition of the egg protein (Block and Mitchel, 1946).

All estimated indices are statistically higher (P<0.05) for the BYL sample than for the cBYL control (Table 8). In particular, Protein Score and Biological Value are 15% higher at the end of fermentation compared to the control matrix. The Nutritional Index, the only index that takes into account, in its calculation, in addition to the qualitative parameters (essential amino acids of the protein) also the amount of bioavailable protein, is 63% higher in BYL than cBYL.

TABLE 8
Chemical score and nutritional indices relating to the digestible
protein fraction of the control (cBYL) and of the base yoghurt-
like food composition (BYL-base). Each value was expressed
as the mean ± standard deviation (n = 3).

| Chemical Score | cBYL | BYL-base |
|---|---|---|
| Histidine | 85 ± 1 | 92 ± 1 |
| Isoleucine | 65 ± 1 | 64 ± 1 |
| Leucine | 88 ± 1 | 96 ± 2 |
| Lysine | 113 ± 1 | 114 ± 1 |
| Cystine | 42 ± 2 | 55 ± 1 |
| Methionine | 38 ± 1 | 44 ± 2 |
| Phenylalanine + Tyrosine | 49 ± 1 | 63 ± 1 |
| Threonine | 78 ± 1 | 78 ± 1 |
| Valine | 69 ± 1 | 70 ± 1 |
| Tryptophan | 44 ± 1 | 62 ± 1 |
| Protein score | 38 ± 1 | 44 ± 2 |
| Biological Value (BV) | 57 ± 2 | 66 ± 2 |
| Protein Efficiency Ratio (PER) | 33 ± 1 | 36 ± 2 |
| Essential Amino Acid Index (EAAI) | 63 ± 2 | 71 ± 2 |
| Nutritional Index (NI) | 1.70 ± 0.08 | 2.77 ± 0.11 |

### Hydrolysis Analysis of Starch

BYL was characterized for starch hydrolysis ratio through an assay protocol mimicking in vivo starch digestion. The ratio of digested starch, the value of the hydrolysis index (HI, Hydrolysis Index) and the relative calculation of the predicted glycemic index (pGI, predicted glycemic index) are shown in Table 9. Specifically, after 180 minutes of in vitro digestion, the percentage of hydrolyzed starch is 38% in the cBYL control, while it decreases in a statistically significant way to a value of 25% in the BYL object of the present invention. To confirm the data, in the BYL the pGI value is reduced by 7% compared to the control. The decrease in HI in BYL is associated with the high fiber content in the food and the biological acidification process carried out by lactic acid bacteria (De Angelis et al., 2007).

TABLE 9
Starch hydrolysis index (HI, hydrolysis index) and predicted glycemic
index (pGI) relative to the comparison composition (cBYL) and
the yoghurt-like base (BYL-base) food composition. Each value
was expressed as the mean ± standard deviation (n = 3).

| Samples | HI (%) | PGI (%) |
|---|---|---|
| cBYL | 38.2 ± 0.2 | 60.6 ± 0.5 |
| BYL-base | 25.1 ± 0.4 | 53.4 ± 0.8 |

### Total Polyphenols and Antioxidant Activity

The concentration of polyphenols in the BYL was equal to 0.28±0.03 mmol (equivalent of gallic acid)/L. The presence of polyphenols in food is of great interest for the antioxidant and anti-inflammatory properties that follow (Covas et al., 2006. The effect of polyphenols in olive oil on heart disease risk factors: a randomized trial. Ann Intern Med, 145:333-41). To evaluate the antioxidant properties, a protocol based on the scavenging activity on the synthetic radical DPPH was used. After 10 minutes of reaction, the scavenging activity on DPPH in the cBYL control was equal to 37.1±0.8%, while in BYL it had increased to a value of 46.2±0.4% (Table 10).

TABLE 10
Concentration of total polyphenols and antioxidant activity
determined in the cBYL control and in the base yoghurt-
like food composition BYL. Each value was expressed
as the mean ± standard deviation (n = 3).

| | Total phenols mmol | Scavenging |
|---|---|---|
| Sample | (gallic acid eq.)/L | activity on DPPH |
| cBYL | 0.19 ± 0.02 | 37.1 ± 0.8 |
| BYL-base | 0.28 ± 0.03 | 46.2 ± 0.4 |

### Concentration in Anti-Nutritional Factors

Cereals, legumes and pseudocereals contain, albeit in a different way depending on the species considered, compounds capable of altering, by reducing it, the bioavailability of some nutrients and the digestibility of food matrices. In order to quantify the reduction of anti-nutritional factors resulting from the biotechnological preparation processes of the yoghurt-like base food composition (mainly related to fermentation), the concentrations of the same were determined in cBYL and BYL. Specifically, as can be seen in Table 11, the biotechnological process, including fermentation with lactic acid bacteria L. plantarum DSM 33326 and L. brevis DSM 33325, allows a reduction of anti-nutritional factors, compared to a matrix obtained with the same ingredients, but in the absence of the fermentation process guided with strains of selected lactic acid bacteria, equal to about 90% for tannins; about 40% for raffinose; about 50% for phytic acid, about 30% for saponins.

TABLE 11
Concentration (mg/100 ml) in anti-nutritional factors in the cBYL
control and in the yoghurt-like base food composition BYL. Each
value was expressed as the mean ± standard deviation (n = 3).

Anti-nutritional factor (mg/100 mL)

| | cBYL | BYL-base |
|---|---|---|
| Tannins | 1.6 ± 0.1 | 0.2 ± 0.2 |
| Raffinose | 18.0 ± 0.2 | 10.7 ± 0.1 |
| Phytic acid | 69.0 ± 0.3 | 35.5 ± 0.9 |
| Saponins | 47.8 ± 0.7 | 32.9 ± 0.7 |

### Example 4. Sensory Characteristics and Shelf Life of the Base Yoghurt-Like Food Composition (BYL-Base) a Flavored Variant, a Probiotic Variant, and the Unfermented Control Composition (cBYL) Materials and Methods

### Samples Analyzed

The base yoghurt-like food composition (BYL-base) and the cBYL control were prepared as described in Example 2.

The BYL-flavored variant and the BYL-probiotic variant were prepared as described in example 2 and contain, respectively, 25% of strawberry puree (humidity 35%), additional ingredient (E), added at the end of the 12 hours of fermentation required by the production protocol and 10⁹ cfu/ml of live and viable cells of the probiotic microorganism Lactobacillus rhamnosus SP1, additional ingredient (H) added in freeze-dried form at the end of the 12 hours of fermentation required by the production protocol,

### Sensory Analysis

The sensory analysis was carried out through panel tests with expert tasters at the Microbiology section of the Department of Soil, Plant and Food Sciences of the University of Bari. The analysis has provided for the comparison of BYL-base and BYL-flavored with cBYL control. Sensory attributes were defined and discussed in a preliminary panel meeting (training session) (Lorusso et al., 2018). The panel consisted of 10 tasters (5M, 5F; average age 27). Sensory attributes were rated on an intensity scale between 0 and 10 (0, not perceived; 2-4 perceived; 5-6 perceived with medium intensity; 7-8 intense; 9-10 very intense). The attributes used were the following:
odor (general intensity, sour, fruit, creamy);
taste (sweet, salty, bitter, sour, astringent);
aftertaste (sweet, salty, earthy);
texture (uniformity, graininess, viscosity, adherence to the spoon);
color (general intensity),

### Microbiological Shelf Life, Lactic Acid Bacteria Survival and Probiotic Microorganism Survival

Lactic acid bacteria were enumerated as described in the materials and methods section of Example 1. The starters were enumerated on Sabouraud Dextrose Agar (SDA, Oxoid, Basinstoke, Hampshire, United Kingdom), containing 150 ppm of chloramphenicol, for 48 hours at 25° C. Molds were enumerated on Potato Dextrose Agar (PDA, Oxoid), for 48 hours at 25° C. Enterobacteriaceae were determined on Violet Red Bile Glucose Agar (VRBGA, Oxoid) for 24 hours at 37° C.

To monitor the presence of Lactobacillus brevis DSM 33325 and Lactobacillus plantarum DSM 33326 starters and probiotics, analyzes were carried out by RAPD-PCR. DNA was extracted from colonies from plates with the highest dilution in MRS and subsequently used for RAPD-PCR analysis (Minervini et al., 2009. Fermented goats' milk produced with selected multiple starters as a potentially functional food. Food Microbiology. 26: 559-64). The analysis by RAPD-PCR was performed as previously proposed by Coda et al. (Coda, et al., 2010. Spelt and emmer flours: characterization of the lactic add bacteria microbiota and selection of mixed starters for bread making. Journal of Applied Microbiology, 108: p. 925-35), using primers P7, and M13—(Invitrogen, Milan, Italy) (De Angelis et al., 2006 Selection of potential probiotic lactobacilli from pig feces to be used as additives in pelleted feeding. Research in Microbiology. 157: 792-801).

### Results

### Sensory Profile

The sensory profile of BYL-base and the corresponding “flavored” formulation is significantly different from the control cBYL (Table 12). BYL-base and BYL-flavored show an intense and complex odor compared to cBYL control. In particular, the BYL-base is characterized by a pleasant sour smell. As expected, the BYL-flavored has a fruity connotation of the olfactory profile. With regard to the flavor, BYL-base was evaluated as the formulation with the highest sour perception. In BYL-flavored, the sour taste is attenuated in favor of the sweet taste. The aftertaste appears to be almost non-existent in all the samples, except in the flavored BYL where the sweetness is perceived. The best texture appears to be attributed to the BYL-base and BYL-flavored, confirming the fact that also the consistency, as well as the gustatory/olfactory notes, improve as a result of the fermentation process. Finally, the BYL-flavored turns out to have a more intense color, and totally dependent on the ingredient added fruit, compared to BYL-base, characterized by creamy white color.

TABLE 12
Sensory profile of the control (cBYL), of the yoghurt-
like base food composition (BYL-base) and of the flavored
variant (BYL-flavored). Each value was expressed
as the mean ± standard deviation (n = 3).

| | | | |
|---|---|---|---|
| Attributes | cBYL | BYL-base | BYL-flavored |

SMELL

| | | | |
|---|---|---|---|
| General intensity | 2.0 ± 0.5 | 8.5 ± 0.5 | 8.0 ± 0.5 |
| Acidic | 3.0 ± 0.5 | 8.0 ± 1.0 | 5.0 ± 1.0 |
| Fruit | 1.0 ± 0.5 | 1.5 ± 0.5 | 8.5 ± 0.5 |
| Creamy | 5.0 ± 1.0 | 8.0 ± 1.5 | 7.5 ± 1.0 |

TASTE

| | | | |
|---|---|---|---|
| Sweet | 2.0 ± 0.5 | 3.0 ± 0.0 | 8.5 ± 1.0 |
| Salty | 2.5 ± 0.5 | 2.0 ± 0.5 | 2.0 ± 0.5 |
| Bitter | 4.0 ± 0.5 | 2.5 ± 0.5 | 2.5 ± 0.0 |
| Acid | 3.0 ± 0.5 | 8.0 ± 1.0 | 5.5 ± 1.0 |
| Astringent | 2.0 ± 0.5 | 3.5 ± 1.0 | 2.0 ± 0.5 |

AFTERTASTE

| | | | |
|---|---|---|---|
| Sweet | 2.5 ± 0.5 | 2.5 ± 0.5 | 8.5 ± 1.0 |
| Salty | 3.0 ± 0.0 | 2.5 ± 1.5 | 2.5 ± 1.5 |
| Earthy | 2.0 ± 0.5 | 1.5 ± 1.5 | 1.2 ± 1.0 |

TEXTURE

| | | | |
|---|---|---|---|
| Uniformity | 6.0 ± 1.0 | 7.5 ± 1.0 | 8.5 ± 0.5 |
| Graininess | 4.0 ± 0.5 | 3.0 ± 1.0 | 3.0 ± 0.5 |
| Viscosity | 6.5 ± 0.5 | 8.5 ± 1.0 | 7.0 ± 0.5 |
| Adherence to the spoon | 4.5 ± 0.5 | 9.0 ± 0.5 | 7.5 ± 0.5 |

COLOR

| | | | |
|---|---|---|---|
| General intensity | 2.0 ± 0.5 | 2.0 ± 0.5 | 8.5 ± 0.5 |

### Microbiological Shelf Life and Survival of Starters and Probiotics

The microbiological shelf life was assessed by monitoring molds, yeasts, enterobacteria and the survival of lactic acid bacteria and the probiotic microorganism at the following times: day 0, 10, 20, 30 of storage in refrigerated conditions. As can be seen from the data reported in Table 13, the BYL-base and BYL-probiotic lactic acid bacteria have a stable value over time of living and viable cells, about 9 Log cfu/ml. cBYL has an initial cell density of live and viable lactic acid bacteria cells equal to 2.65±0.10 Log cfu/ml which reaches 5.60±0.34 Log cfu/ml. A complete absence of molds, yeasts and enterobacteria is observed in the BYL and BYL-flavored samples. In cBYL, yeasts are instead present at detectable cell densities, as are molds (the latter over 10 days of refrigerated storage). The differences between the control and the two fermented samples are attributable to the fact that the fermentation process protects the matrix from contamination due to a combined effect of biological acidification, microbial competition by the starter and the production of compounds with antimicrobial activity (organic acids and bacteriocins). In BYL-probiotic, the density of live and viable cells of Lactobacillus rhamnosus SP1 is reduced by a single logarithmic cycle during the entire storage period, changing from 9.36±0.32 per day 0 to 8.12±0.13 Log cfu/ml on day 30.

TABLE 13
Microbiological analyzes (Log cfu/ml) of the control (cBYL), the
base yoghurt-like food composition (BYL-base) and probiotic variant
(BYL-flavored) during 30 days storage at 4° C. Each value
was expressed as the mean ± standard deviation (n = 3).

| | 0 | 10 | 20 | 30 |
|---|---|---|---|---|
| | c-BYL | | | |
| Lactic acid | 2.65 ± 0.10 | 4.36 ± 0.36 | 4.97 ± 0.54 | 5.60 ± 0.34 |
| bacteria | | | | |
| Entero- | <10 | <10 | <10 | <10 |
| bacteriaceae | | | | |
| Molds | <10 | <10 | 3.11 ± 0.34 | 5.06 ± 0.21 |
| Yeasts | 2.67 ± 0.26 | 3.26 ± 0.14 | 4.24 ± 0.54 | 5.40 ± 0.67 |
| Lb. rhamnosus | — | — | — | — |
| SP1 | | | | |
| | BYL-base | | | |
| Lactic acid | 9.26 ± 0.25 | 9.30 ± 0.41 | 9.03 ± 0.62 | 9.16 ± 0.45 |
| bacteria | | | | |
| Entero- | <10 | <10 | <10 | <10 |
| bacteriaceae | | | | |
| Molds | <10 | <10 | <10 | <10 |
| Yeasts | <10 | <10 | <10 | 3.15 ± 0.23 |
| Lb. rhamnosus | — | — | — | — |
| SP1 | | | | |
| | BYL-probiotic | | | |
| Lactic acid | 9.86 ± 0.25 | 9.10 ± 0.41 | 9.00 ± 0.66 | 9.45 ± 0.40 |
| bacteria | | | | |
| Entero- | <10 | <10 | <10 | <10 |
| bacteriaceae | | | | |
| Molds | <10 | <10 | <10 | <10 |
| Yeasts | <10 | <10 | <10 | <10 |
| Lb. rhamnosus | 9.36 ± 0.32 | 8.87 ± 0.43 | 8.54 ± 0.36 | 8.12 ± 0.13 |
| SP1 | | | | |

### Example 5: Production of a Yoghurt-Like Food Composition, Formulation to Drink (BYL-to Drink) Representative of the Invention and an Unfermented Control Composition (cBYL-to Drink)

### Materials and Methods

### Production Protocol

A BYL to drink representative of the invention was produced as described in Example 2, using the same starting ingredients, i.e. rice/chickpea/lentil in a ratio of 2:1:1 (w/w/w), and resuspending the flours in a different amount of water. In detail, a flour:water ratio of 10:90 (90% vol/w) was used. The different formulation allows obtaining a composition similar, in terms of texture and viscosity, to a conventional “to drink” yoghurt obtained from milk matrix.

The cBYL-to drink control was produced as described in Example 2 without fermenting the matrix but incubating it under the same conditions.

### Microbiological, Chemical-Physical and Nutritional Characterization

BYL-to drink was analyzed for viscosity, nutritional label, pH, lactic and acetic acids, total free amino acids and GABA, total phenols, cell density in lactic acid bacteria at the end of the fermentation process, concentration in anti-nutritional factors (condensed tannins, phytic acid, raffinose and saponins), protein digestibility, starch hydrolysis index HI. The analytical methods used are described in examples 1, 2 and 3.

### Results

BYL-to drink is characterized by a pH, at the end of fermentation, of 4.23±0.12 (pH at the beginning of fermentation 6.53±0.60). The cell density of live and viable cells of lactic acid bacteria at the end of fermentation was equal to 2×10⁹ cfu/ml. The viscosity was equal to 9.21±2.11 mPa·s. The matrix is characterized by lactic and acetic acid concentrations equal to 9.9±0.3 and 4.2±0.1 mmol/L, respectively.

The concentration of TFAA was found to be 636.8±12.5 mg/L. The TFAA concentration in the cBYL-to drink control was 359.2±11.3 mg/L. The concentration of GABA was found to be 67.5±5.2 mg/L (45.5±5.2 mg/L in cBYL-to drink)

The total polyphenol concentration was found to be 0.09±0.01 mmol/L in cBYL-to drink and 0.15 mmol/L in BYL-to drink.

The in vitro digestibility of the proteins was found to be 85.4±0.5 (cBYL to drink was characterized by an IVPD value of 68.2±0.2). The starch hydrolysis index was equal to 22±2 (cBYL-to drink instead, a value equal to 35±2).

The details of the nutritional label are shown in Table 14, while the concentrations of anti-nutritional factors in BYL-to drink and in the respective unfermented control are shown in Table 15.

TABLE 14
Nutritional label of the yoghurt-like composition
in the “to drink” formulation. Each value
was expressed as the mean ± standard deviation (n = 3).

| | 100 g |
|---|---|
| Energy value (Kcal) | 33.8 |
| Fat (g) | 0.29 ± 0.05 |
| Carbohydrates (g) | 6.35 ± 0.18 |
| Fiber (g) | 2.06 ± 0.06 |
| Protein (g) | 1.62 ± 0.19 |
| Ashes (g) | 0.10 ± 0.12 |
| Moisture (g) | 90.2 ± 2.7 |

TABLE 15
Anti-nutritional factors in the control composition (cBYL-to
drink) and in the yoghurt-like composition in the “to
drink” formulation (BYL-to drink). Each value was
expressed as the mean ± standard deviation (n = 3).

Anti-nutritional factor (mg/100 mL)

| | cBYL-to drink | BYL-to drink |
|---|---|---|
| Condensed tannins | 0.8 ± 0.1 | 0.1 ± 0.1 |
| Raffinose | 9.1 ± 0.3 | 4.28 ± 0.6 |
| Phytic acid | 34.5 ± 1.7 | 14.2 ± 0.2 |
| Saponins | 24.2 ± 0.3 | 12.8 ± 0.3 |

### Example 6: Production of a Yoghurt-Like Food Composition, Formulation for Filling (BYL-for Filling) Representative of the Invention and an Unfermented Control Composition (cBYL-for Filling)

### Materials and Methods

### Production Protocol

A BYL-for filling representative of the invention was produced as described in Example 2, using the same starting ingredients, i.e. rice/chickpea/lentil in a ratio of 2:1:1 (w/w/w), but resuspending the flours in a different amount of water. In detail, a flour:water ratio of 30:70 (70% vol/w) was used. The different formulation allows obtaining a composition for fillings that can be used for the preparation of creams, sweet or savory fillings to be used as filling for sweets, sandwiches and other leavened baked products.

### Microbiological, Chemical-Physical and Nutritional Characterization

BYL-for filling was analyzed for viscosity, nutritional label, pH, lactic and acetic acids, total free amino acids and GABA, total polyphenols, cell density in lactic acid bacteria at the end of the fermentation process, concentration in anti-nutritional factors (condensed tannins, phytic acid, raffinose and saponins), protein digestibility, starch hydrolysis index HI. The analytical methods used are described in Examples 1, 2 and 3.

### Results

BYL-for filling is characterized by a pH, at the end of fermentation, equal to 4.2 (pH at the beginning of fermentation 6.53±0.60). The cell density of live and viable cells of lactic acid bacteria at the end of fermentation was equal to 2×10⁹ cfu/ml. The viscosity was equal to approx. 400 Pa·s. The matrix is characterized by lactic and acetic acid concentrations equal to 15.9±0.5 and 6.2±0.1 mmol/L, respectively.

The concentration of TFAA was found to be 1171.2±21.3 mg/L. The TFAA concentration in the cBYL-for filling control was 1055.3±23.3 mg/L. The concentration of GABA was found to be 170.4±2.3 mg/L (125.6±4.3 mg/L in cBYL-for filling)

The total polyphenol concentration in cBYL-for filling was equal to 0.27±0.02 mmol/L, while it was equal to 0.45±0.01 mmol/L in BYL-for filling.

The in vitro digestibility of the proteins was found to be 80.2±1.3 (cBYL to drink was characterized by an IVPD value of 68.5±0.8). The starch hydrolysis index was equal to 27.6±1.2% (cBYL-for filling instead, a value equal to 38.5±0.8%).

The details of the nutritional label are shown in Table 16, while the concentrations of anti-nutritional factors in BYL-for filling and in the respective unfermented control are shown in Table 17.

TABLE 16
Nutritional label of the yoghurt-like composition
in the “for filling” formulation. Each value
was expressed as the mean ± standard deviation (n = 3).

| | 100 g |
|---|---|
| Energy value (Kcal) | 100.8 |
| Fat (g) | 0.81 ± 0.09 |
| Carbohydrates (g) | 19.05 ± 0.10 |
| Fiber (g) | 6.08 ± 0.08 |
| Protein (g) | 4.76 ± 0.07 |
| Ashes (g) | 0.31 ± 0.04 |
| Moisture (g) | 70.5 ± 1.1 |

TABLE 17
Anti-nutritional factors in the control (cBYL-for filling)
and in the yoghurt-like food composition in the “for
filling” formulation (BYL-for filling). Each value
was expressed as the mean ± standard deviation (n = 3).

Anti-nutritional factor (mg/100 mL)

| | cBYL-filling | BYL-filling |
|---|---|---|
| Tannins | 2.51 ± 0.82 | 0.28 ± 0.09 |
| Raffinose | 27.5 ± 0.4 | 14.1 ± 0.3 |
| Phytic acid | 101.5 ± 1.3 | 46.8 ± 0.5 |
| Saponins | 70.6 ± 0.9 | 42.2 ± 1.1 |

### Example 7: Production of a Dehydrated Food Composition (BYL-Lyophile) Representative of the Invention

### Materials and Methods

### Production Protocol

A BYL-lyophile was produced by preparing the base yoghurt-like food composition (BYL-base) as described in Example 2, and subsequently freeze-drying such a composition with conventional methods known to those skilled in the art.

### Microbiological, Chemical-Physical and Nutritional Characterization

The lyophile was analyzed for nutritional label, total free amino acids and GABA, total polyphenols and antioxidant activity, cell density in lactic acid bacteria, concentration in anti-nutritional factors (condensed tannins, phytic acid, raffinose and saponins), digestibility of proteins. The analytical methods are described in examples 1, 2 and 3.

### Results

The lyophile was characterized by a cell density of lactic acid bacteria equal to 2×10¹⁰ cfu/g, a concentration of TFAA equal to 5314.3±27.8 mg/kg and of GABA equal to 495.8±8.5 mg/kg. The total polyphenol concentration was found to be 1.55±0.07 mmol/kg, the antioxidant activity of the methanol extract (scavenging activity on DPPH radical) was equal to 80±3%.

In vitro digestibility of proteins was found to be 80.5±1.2%.

The details of the nutritional label are shown in Table 18, while the concentrations of anti-nutritional factors are shown in Table 19.

TABLE 18
Nutritional label of the dehydrated food composition
(freeze-dried). Each value was expressed as the
mean ± standard deviation (n = 3).

| | 100 g |
|---|---|
| Energy value (Kcal) | 306 |
| Fat (g) | 2.20 ± 0.10 |
| Carbohydrates (g) | 56.5 ± 0.80 |
| Fiber (g) | 18.2 ± 0.51 |
| Protein (g) | 14.2 ± 0.07 |
| Ashes (g) | 0.91 ± 0.07 |
| Moisture (g) | 10.2 ± 0.9 |

TABLE 19
Anti-nutritional factors in the dehydrated food
composition (freeze-dried). Each value was expressed
as the mean ± standard deviation (n = 3).

| | Anti-nutritional factor (mg/100 g) |
|---|---|
| | Lyophile |
| Tannins | 0.91 ± 0.02 |
| Raffinose | 49.5 ± 1.3 |
| Phytic acid | 159.7 ± 1.7 |
| Saponins | 144.5 ± 4.7 |

As can be seen from the data reported in the above experimental part, the food composition according to the present invention is produced exclusively starting from vegetable ingredients and can be classified as “vegan”, in particular it does not contain milk or other ingredients containing lactose. This composition is produced exclusively with naturally gluten-free ingredients, therefore it does not contain gluten and is commercially classifiable as a gluten-free food (Commission Implementing Regulation (EU) No. 828/2014 of 30 Jul. 2014). Furthermore, such a composition is produced with and contains lactic acid bacteria, preferably of the Lactobacillus plantarum and Lactobacillus brevis species, recognized as QPS (Qualified Presumption of Safety) by EFSA (Ricci et al., 2017. Scientific Opinion on the update of the list of QPS-recommended biological agents intentionally added to food or feed as notified to EFSA. EFSA Journal, 15:1-178). The lactic acid bacteria contained in the food composition of the present invention are alive and viable and have cell density greater than 100,000,000 cells/ml (10⁸ cfu/ml), which persists for at least 30 days in refrigerated conditions (4° C.), see Table 13. To maintain and preserve the food composition and the organoleptic profile of the present invention intact, it should preferably be stored at a temperature in the range between 2° and 8° C. until the moment of consumption.

In particular, as shown in Table 6, the base yoghurt-like food composition (BYL-base) contains a proportion of fibers equal to about 4.10±0.20% with respect to the total weight of the composition, therefore this food can be defined as “high in fiber” (according to Regulation (EC) No. 1924/2006 of the European Parliament and of the Council of 20 Dec. 2006 on nutrition and health claims made on food); it is possible to bring this concentration to 6% with respect to the total weight of the composition if one or more vegetable ingredients with a high fiber content (G) are used.

As shown in Table 6, the base composition (BYL-base) contains a percentage of proteins equal to about 3.2% with respect to the total weight of the composition, therefore this food can also be defined as “high protein content” (according to the Regulation (EC) mentioned above); it is possible to bring this percentage to 5-6% with respect to the total weight of the composition if one or more vegetable ingredients with a high protein content (G′) are used.

As shown in Table 7, the base composition (BYL-base) is characterized by a high concentration in total free amino acids (TFAA) greater than 1000 mg/L. These amino acids released during the fermentation process represent an index of the proteolytic activity of the studied strains, which in turn it is to be considered an important qualitative parameter since it is related to the sensory and nutritional characteristics of the product. Furthermore, Table 7 also shows the concentration of the functional amino acid GABA (γ-aminobutyric acid) higher than 100 mg/L).

As shown in example 3, in relation to the IVDP value, the base composition (BYL-base) is characterized by a high digestibility of proteins, that is, greater than 75%, thanks to the fermentation process carried out with the selected lactic acid bacteria. As shown in Table 8, with regard to the digestible protein fraction, this composition has high nutritional indices: Protein Score higher than 40; Protein Efficiency Ratio PER, greater than 30; essential amino acids index EAAI, greater than 65; Nutritional Index, NI, greater than 2.0.

As shown in Table 9, the base yoghurt-like food composition (BYL-base) is characterized by a predicted glycemic index less than 55, this value is linked to the high fiber content and biological effect of acidification carried out by the selected lactic acid bacteria.

As shown in Table 10, the base yoghurt-like food composition (BYL-base) is characterized by the presence of polyphenols in a concentration higher than 0.2 mmol (gallic acid equivalent)/L, and shows high antioxidant activity, in part due to native properties of the ingredients used and partly as a consequence of the fermentation process carried out with the selected lactic acid bacteria. The concentration of polyphenols can reach values in the range 0.4-1.8 mmol/L if matrices are used (e.g. fruit and derivatives as per point (E) of the ingredients list).

As shown in Table 11, the base yoghurt-like food composition (BYL-base) is characterized by a reduced presence of anti-nutritional compounds present in the raw materials, such as condensed tannins, raffinose (and other alpha-galactosides), phytic acid, and saponins, thanks to the fermentation process carried out with the selected lactic acid bacteria.

As shown in Table 5, the texture of the base yoghurt-like (BYL) food composition is similar to that of a conventional yoghurt; as reported in example 5, the texture of the yoghurt-like food composition to drink (CYL-to drink) is similar to that of a conventional yoghurt to drink.

It is specified that viscosity is a characteristic that the matrix acquires in the heat treatment step (pre-fermentative) and depends on the amount of water used for the preparation of the matrix. Consequently, as shown in the following Table 20, the ranges of the % nutritional values of the compositions BYL, BYL-to drink and BYL-for filling are different from each other.

TABLE 20
Nutrition label of base yoghurt-like food composition (BYL-base)
of the yoghurt-like to drink food composition (BYL-to drink and
yoghurt-like food composition for filling (BYL-for stuffing).

| | BYL-base | BYL-to drink | BYL-for filling |
|---|---|---|---|
| Fats (%) | 0.4-0.6 | 0.2-0.4 | 0.7-1.1 |
| Carbohydrates (%) | 10-15 | 5-10 | 15-22 |
| Fibers (%) | 3.5-5.5 | 1.5-3.0 | 5.0-9.0 |
| Proteins (%) | 2.8-4.0 | 1.3-2.5 | 4.0-6.0 |
| Ashes (%) | 0.20-0.40 | 0.08-1.8 | 0.25-0.45 |
| Humidity (%) | 76-84 | 86-94 | 66-74 |

In light of these results, all the tested compositions representative of the invention are characterized by possessing both excellent functional features and solid nutritional bases.

Furthermore, as shown in Table 12, all the tested compositions representative of the invention have excellent sensory features.

Finally, as shown in Tab.13, all the tested compositions representative of the invention are characterized by an excellent microbiological shelf life.

1. Bacterial strain selected from:
Lactobacillus brevis, filed on 14 Nov. 2019 with Deutsche Sammlung von Mikroorganismen und Zellkulturen GmbH and identified with the filing number DSM 33325, and
Lactobacillus plantarum, filed on 14 Nov. 2019 with Deutsche Sammlung von Mikroorganismen und Zellkulturen GmbH and identified with the filing number DSM 33326.

2. (canceled)

3. Food composition based on at least one gluten-free cereal flour (A) and/or at least one legume flour (B), water (C) and a starter (D) comprising Lactobacillus brevis DSM 33325 and/or Lactobacillus plantarum DSM 33326, said components (A) and/or (B) being fermented with said starter (D), and
optionally comprising one or more additional gluten-free ingredients selected from: one or more fruit-based ingredients (E), and/or one or more vegetable ingredients (F), and/or one or more vegetable ingredients with a high fiber content (>50% w/w) (G) or high protein content (>50% w/w) (G′), and/or one or more viable probiotic microorganisms (H), and/or at least one salt and/or flavor enhancer (I), and/or at least one sugar and/or sweetener (J), and/or at least one structuring agent (K).

4. Food composition according to claim 3, wherein
said gluten-free cereal flour (A) is selected from naturally gluten-free cereal flours
and/or
said legume flour (B) is selected from flours of bean, Phaseolus vulgaris L.; pea, Pisum sativum L.; broad bean, Vicia faba L.; lupine, Lupinus albus; chickpea, Cicer arietinum L.; pigeon pea, Cajanus indicus; peanuts, Arachis hypogaea L.; soy, Glycine max; lentil, Lens culinaris; cicerchia, Lathyrus sativus; carob, Ceratonia siliqua; pseudocereals (amaranth, Amaranthus spp., quinoa, Chenopodium quinoa; buckwheat, Fagopyrum esculentum) and mixtures thereof.

5. Food composition according to claim 3, wherein said composition has a cell density greater than 10⁸ cfu/ml.

6. Food composition according to claim 3, wherein said composition comprises a concentration in total free amino acids (TFAA) greater than 800 mg/L.

7. Food composition according to claim 3, wherein said composition comprises a concentration of the functional amino acid GABA (γ-aminobutyric acid) greater than 100 mg/L.

8. Food composition according to claim 3, wherein said composition comprises less than 1 mg of tannins, calculated on 100 mL of food composition.

9. Food composition according to claim 3, wherein said composition comprises less than 15 mg of raffinose, calculated on 100 mL of food composition.

10. Food composition according to claim 3, wherein said composition comprises less than 50 mg of phytic acid, calculated on 100 mL of food composition.

11. Food composition according to claim 3, wherein said composition comprises less than 40 mg of saponins, calculated on 100 mL of food composition.

12. Food composition according to claim 3, wherein said composition comprises a concentration of polyphenols equal to or greater than 0.050 mmol/L, expressed as mmol/L of equivalent gallic acid.

13. Food composition according to claim 3, wherein said composition comprises a concentration of polyphenols equal to or greater than 0.20 mmol/L, expressed as mmol/L of equivalent gallic acid.

14. Food composition according to claim 3 in yoghurt-like form (BYL), having a viscosity of between 0.001 and 1,000 Pa·s measured as described in the experimental part.

15. Food composition according to claim 3 in dehydrated form (BYL-dehydrated).

16. (canceled)

17. Process for the production of a food composition, comprising the following steps:
a) mixing in water (C) at least one gluten-free cereal flour (A) and at least one legume flour (B), obtaining a mixture;
b) subjecting the mixture to heat treatment (gelatinization) at a temperature between 65° and 100° C., obtaining a gelatinized mixture;
c) cooling the gelatinized mixture to a temperature between 2° and 8° C.,
d) inoculating into the cooled gelatinized mixture obtained in step c), after heating at a temperature between 20° and 40° C., a starter (D) comprising at least one lactic acid bacterium selected from the following species Lactobacillus brevis, Lactobacillus plantarum, Pediococcus acidilactici, Leuconostoc mesenteroides, Lactobacillus rossiae, and mixtures thereof,
e) fermenting the mixture resulting from step d) at a temperature between 20° and 40° C.,
f) cooling the mixture resulting from step e) to a temperature between 2° and 8° C., and
optionally comprising the following steps
g) high-pressure homogenizing, and/or
h) adding further ingredients, suitably pasteurized if necessary, such as one or more fruit-based ingredients (E), and/or one or more vegetable ingredients (F), and/or one or more gluten-free vegetable ingredients with a high fiber content (>50% w/w) (G) or high protein content (>50% w/w) (G′), and/or one or more viable probiotic microorganisms (H), and/or at least one salt and/or flavor enhancer (I), and/or at least one sugar and/or sweetener (J), and/or at least one structuring agent (K), and/or
i) packaging, and/or
j) dehydrating by freeze-drying or spray-drying.

18. Process for the production of a food composition according to claim 17, wherein said starter (D) comprises Lactobacillus brevis DSM 33325 and/or Lactobacillus plantarum DSM 33326.

19. Process for the production of a food composition according to claim 17, wherein said starter (D) comprises Lactobacillus brevis DSM 33325 and Lactobacillus plantarum DSM 33326 in a cell density ratio between 1:1 and 1:10.

20. (canceled)

21. Food composition according to claim 3, wherein the starter (D) comprises Lactobacillus brevis DSM 33325 and Lactobacillus plantarum DSM 33326 in a cell density ratio between 1:1 and 1:10.

22. Food composition according to claim 5, having a cell density between 1×10⁹ and 7×10⁹ cfu/ml.

23. Food composition according to claim 14, having a viscosity of between 2-15 Pa·s (BYL-base), or between 5-50 mPa·s (BYL-to drink), or between 100-800 Pa·s (BYL—for filling) measured as described in the experimental part.
